(19) United States
(12) Patent Application Publication
(10) Pub. No.: US 2014/0088578 A1
(43) Pub. Date: Mar. 27, 2014

(54) CRYOELECTRIC SYSTEMS AND METHODS FOR TREATMENT OF BIOLOGICAL MATTER

(71) Applicant: THE REGENTS OF THE UNIVERSITY OF CALIFORNIA, Oakland, CA (US)

(72) Inventors: Boris Rubinsky, El Cerrito, CA (US); Charlotte Daniels, Berkeley, CA (US); Yair Granot, Albany, CA (US); Liel Rubinsky, El Cerrito, CA (US)

(73) Assignee: THE REGENTS OF THE UNIVERSITY OF CALIFORNIA, Oakland, CA (US)

(21) Appl. No.: 14/035,866

(22) Filed: Sep. 24, 2013

Related U.S. Application Data
(63) Continuation of application No. PCT/US2012/031728, filed on Mar. 30, 2012.
(60) Provisional application No. 61/470,718, filed on Apr. 1, 2011.
(60) Provisional application No. 61/493,460, filed on Jun. 5, 2011.

(51) Int. Cl. A61B 18/02

(52) U.S. Cl. CPC A61B 18/02
USPC 606/21

(57) ABSTRACT

Systems and methods are disclosed for treating a target volume of biological matter, by cooling a volume of tissue to a temperature below freezing, and directing an electric field through the cooled volume of tissue or tissue adjacent to the cooled volume of tissue to generate at least a temporary physiological affect on one or more of the cooled volume of tissue and adjacent volume of tissue. The generated physiological affect may include shielding a region of tissue from treatment, focusing treatment on a particular region of tissue, and sterilization of tissue.

### CROSS-REFERENCE TO RELATED APPLICATIONS

This application is a 35 U.S.C. §111(a) continuation of PCT international application number PCT/US2012/031728 filed on Mar. 30, 2012, incorporated herein by reference in its entirety, which is a nonprovisional of U.S. provisional patent application Ser. No. 61/470,718 filed on Apr. 1, 2011, incorporated herein by reference in its entirety, and a nonprovisional of U.S. provisional patent application Ser. No. 61/493,460 filed on Jun. 5, 2011, incorporated herein by reference in its entirety.

The above-referenced PCT international application was published as PCT International Publication No. WO 2012/135786 on Oct. 4, 2012 and republished on Nov. 22, 2012, and such publications are incorporated herein by reference in their entirety.

### STATEMENT REGARDING FEDERALLY SPONSORED RESEARCH OR DEVELOPMENT

This invention was made with Government support under Grant Number R01RR018961-03 awarded by the National Institutes of Health. The Government has certain rights in the invention.

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to cryoelectric treatment applications, and more particularly to the combined use of freezing and electric fields on biological matter for applications in the food industry, biotechnology and medicine.

2. Description of Related Art

During the last 20 years the development and implementation of minimally and non-invasive surgeries have flourished. In comparison to traditional surgery, minimally and non-invasive surgeries are positioned to transform the field of medicine with shorter hospital stays, reduced surgical trauma, improved immune response and greater precision. These benefits are primarily due to less intrusive procedures and more targeted tissue ablation. Among the primary physiological effects used for selective ablation of undesirable tissues are a) thermal effects on molecules in living biological matter and b) electric and electromagnetic effects on molecules in living matter. Tissue ablation by “thermal effects” employs non physiological temperatures to destroy tissues by affecting the chemical structure of molecules exposed to those non-physiological temperatures. Temperature related ablation modalities can be categorized as of two types: (1) temperatures above, and (2) temperatures below, physiological temperature.

Thermal ablation techniques that utilize above physiological temperatures consist of technologies that deliver energy to the targeted volume of tissue for the purpose of elevating the temperature to non physiological levels. The technologies include ultrasound, and various electric such as: radiofrequency (RF), microwave (MW), DC currents and AC currents. The mechanism involved in tissue ablation through temperature elevation with electric and electromagnetic fields employ the Joule effect, which consists of electrical energy dissipation in the targeted tissue.

Cryosurgery is the name of the technology used for the thermal ablation methods that employ temperatures below freezing temperatures. The fundamental science of cryosurgery can be found in the field of cryobiology. Cryobiology is the field of life sciences that deals with the effect of temperatures below the freezing temperature on biological matter. Fundamental research in cryobiology finds that freezing has a well known paradoxical effect on biological matter. On one hand, it can preserve biological matter in an application known as “cryopreservation,” and on the other hand, it can destroy biological matter in the application known a “cryosurgery.”

The particular effect of freezing, whether it destroys or preserves biological matter, is a function of many parameters, such as: cooling and heating rates during freezing and thawing, minimal temperature during freezing, time of storage and the presence of various chemical additives in tissue. The biophysical mechanism that leads to this paradoxical effect is well understood and has been studied extensively. It is related to the thermodynamics of freezing in solutions and to the mass transfer during freezing of cells.

The preserving effect of low temperature is commonly used for long-term storage of cells and tissues. For example, freezing is commonly used for long-term preservation of sperm and oocytes while cold is used for preservation of hearts prior to transplantation.

Low temperatures also have the ability to reduce the metabolism, e.g. the lower the temperature the lower the metabolism. Therefore, another use of low temperatures, in particular temperatures below freezing, is for freezing storage as a way to reduce the proliferation of microorganisms. This is the principle behind refrigeration of food and long-term storage in a frozen state. However, during preservation of foods by freezing, detrimental microorganisms can survive the high subzero freezing temperatures used in home refrigerators and will proliferate upon thawing.

Currently, there are no simple methods for sterilization of frozen products. Therefore, the transition from a sterilized unfrozen product to a frozen product is a substantial source of contamination. Freezing, in particular at high subzero temperatures, does not destroy most biological contamination. Rather, freezing only reduces metabolism. This is why frozen products cannot be refrozen and reused. Such an occurrence leads to severe health consequences for users of frozen foods.

The destructive effect of freezing is used for the minimally invasive surgical modality known as cryosurgery. In cryosurgery, a cryosurgical probe that is often a hollow probe cooled with a cryogen induces the freezing. FIG. 1 shows a cryosurgery probe 10 inserted in the center of the undesirable tissue 18. To achieve low cryogenic temperatures (e.g. −160° C.), the probe 10 directs liquid nitrogen N₂ from proximal end 26 down a central channel 22 to a boiling chamber 28 at distal tip 20, in which the liquid nitrogen boils and produces N₂ gas that is directed back proximally out the probe 10. Insulation walls 24 focus the cooling at the distal end 20. The outer walls of the cryosurgical probe 12 are made of metal, such as stainless steel.

The freezing interface propagates, as a function of time, from the distal probe surface 20 outward into the tissue. The hope is that the part of the frozen lesion 14 in which cells die will encompass the entire undesirable tissue and that the freezing will be with such thermodynamic parameters that will destroy the undesirable tissue. As shown in FIGS. 3A and 3B, cells survive freezing in the temperature range from −0.56° C. and about −30° C., e.g. the region from 16 to 14 shown in FIG. 1.

Another problem with the device 10 of FIG. 1 is with respect to detecting the extent of the frozen region 14, which being inside the tissue 18 is not visible to the naked eye. While intraoperative imaging has been developed to determine the extent of freezing deep inside tissue, such imaging provides information on the outer margin 16 of the frozen lesion only, i.e. the interface between frozen and the adjacent unfrozen tissue. As explained above, and shown in FIGS. 3A and 3B, the interface 16 between frozen and unfrozen tissue does not correspond to the extent of complete tissue death, as the frozen region between isotherms 16 and 14 contains cells that survive freezing. Thus, while it is possible to image the extent of freezing in real time, the extent of freezing that is imaged does not correspond to the extent of tissue death—for which there is no control.

In cryosurgery, it is known that freezing produces the ability of the cryosurgical probe to attach tightly to the frozen tissue. This occurs at high subzero temperatures, as soon as freezing begins, and is used to attach the cryoprobe to the desired location in tissue without any other means. In typical cryosurgery systems, setting the cryosurgical probe to the “stick” mode produces this. This mode yields excellent contact between the probe and the tissue and will also be used in the applications of this invention combining freezing with electric fields.

The effects of electric and electromagnetic fields on molecules in living matter are becoming commonly used for food sterilization, biotechnology and medicine. The relevant technology known by different names such as “pulsed electric fields” (PEF), or electroporation, or nanosecond pulses utilizes electric fields that target cell molecules such as the cellular membrane, increasing membrane permeability through the formation of nanoscale defects in the membrane. The effect of these electric fields on cells is also known as electroporation, or electropermeabilization. Electroporation or electropermeabilization is a significant increase in the electrical conductivity and permeability of the cell plasma membrane caused by an externally applied electrical field. The effect is presumably related to defects that form in the cell membrane. These defects can be temporary and “reversible” and then they are used in biotechnology and medicine to insert chemical compounds that normally do not enter the cell membrane into cells. The defects can also be of the permanent type, “irreversible,” where they are used to destroy cells in food sterilization and directly ablate cells in minimally invasive surgery. The destructive effect of certain electric fields (EF) can be also accomplished through their effect on other parts of the cell, such as the DNA. The electric fields (EF's) can be delivered through electrodes in contact with the tissue, although they can also be delivered in a non-contact way through capacitance and inductive effects. The irreversible electroporation use of PEF has emerged as a minimally invasive technique for tissue ablation, because of its molecular selectivity.

While each has their distinct benefits, the application of cryotherapy and electric fields induced electroporation in combination has not been studied, particularly because the heating of tissue often associated with the administration of electric fields would be counterproductive to the ablation of tissue by cooling typically associated with cryotherapy.

### BRIEF SUMMARY OF THE INVENTION

The systems and methods of the present invention exploit the combined use of freezing and electroporation inducing electric fields on biological matter. The use of these freezing and electric fields in certain combinations gives rise to effects on biological matter that cannot be achieved with either freezing or electroporation inducing electric fields separately, with utility in many applications.

Freezing and low temperatures have a paradoxical effect on biological tissues. They can, as a function of the biophysical processes that occur during cooling and warming, protect living cells or destroy living cells.

Electric fields of the type that affect living cells have also a paradoxical effect. They can, as a function of the biophysical processes that electric fields induce on cells, cause reversible permeabilization of the cell membrane, which is used in biotechnology and drug therapy. Or, they can lead to irreversible damage to living cells through various effects on the cell from thermal to effects on various components of the cell, which has applications ranging from the sterilization of foods and for minimally invasive surgical tissue ablation.

The systems and methods of the present invention utilize the relation between temperature and tissue thermodynamic state and electrical impedance of tissue to combine two different fields: cryobiology and electroporation inducing electric fields in tissue. The temperature dependent electrical properties of tissue have a significant impact when cryobiology and electric fields are used together to accomplish useful applications.

The systems and methods of the present invention take advantage of modifications in the electrical circuit that forms when the electrical properties of tissue are modulated by temperature. The changes in electrical impedance of tissue, with temperature in general and freezing in particular, are specifically configured with the combined use of cold and freezing and electric fields.

In a first embodiment, applying electric fields across frozen physiological solutions is used for destruction of microorganisms that survive high subzero freezing temperatures during storage.

In a second embodiment, application of both electric fields and cooling are delivered from the same source (e.g. a cryosurgical probe connected to a cryogen source and an EF delivery source) such that the electroporation inducing electric field can be used to destroy cells that survive freezing in the frozen lesion, in addition to confining the electroporation inducing electric fields to the frozen lesion and the surrounding tissue. This facilitates, for example, using irreversible PEF or electro chemotherapeutic PEF tissue ablation to destroy cells frozen at high subzero temperatures at which cells normally survive freezing. Thereby, imaging the extent of tissue ablation through imaging of the frozen lesion ensures that the cells in the frozen lesion are destroyed by the combination of EF and freezing to at least the margin of the frozen lesion.

In a third embodiment, electric fields are applied from a different source than the freezing source such that the electric fields in the frozen lesion are negligible and have no effect on the frozen lesion. Therefore, in this configuration, freezing applied with cryoprotective conditions on a target volume of tissue in combination with electroporation inducing PEF's to the entire volume of interest, e.g. including the frozen lesion, the frozen area will survive the combined application of freezing and pulsed electric fields. This configuration has particular value in applications in which it is desired to spare parts of the electroporation treated tissue from the electroporation effects—for the bladder sphincter in the PEF treatment of the prostate.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a schematic diagram of a cryosurgical probe deployed in tissue and resulting ice ball that forms around the tip of the probe inside the tissue.

FIG. 2 is a plot of impedance on a log scale in ohms, as a function of temperature in a physiological saline solution during freezing (dashed line) and thawing (solid line).

FIGS. 3A and 3B are plots showing the effect of temperature and cooling rate on cell death in ND-1 prostate cancer cells. FIG. 3A shows cells frozen with a cooling rate of 5 C/min, and FIG. 3B shows cells frozen with a cooling rate of 25 C/min.

FIGS. 4A-C illustrate three fundamental electrical circuit designs of a combination frozen and unfrozen tissue in accordance with the present invention.

FIG. 5 shows a schematic diagram of a sterilization system configured for applying EF to a uniformly frozen biological matter in accordance with the present invention.

FIG. 6 shows a schematic diagram of a cryoelectric system in accordance with the present invention.

FIG. 7A-D show a test setup for the cryoelectric system of FIG. 6.

FIG. 8 illustrates a schematic diagram of a shielding cryosurgery system in accordance with the present invention.

FIG. 9A-C show a test setup for the cryosurgery/EF system of FIG. 8.

FIGS. 10A and 10B illustrate cryoelectric probes used in a stick mode for coupling to tissue in accordance with the present invention.

FIG. 11 shows an electric current schematic for a one dimensional test case in accordance with the present invention.

FIG. 12A shows a finite element mesh for a second test case in which one cryoelectric probe is positioned in the center of tissue cylinder.

FIG. 12B shows a finite element mesh for a third test case in which two probes are positioned within the tissue.

FIGS. 13A and 13B show graphs for temperature and electric field distribution, respectively, during 90 seconds of freezing.

FIGS. 13C and 13D show graphs for temperature and electric field distribution, respectively, during 90 seconds of thawing.

FIGS. 14A and 14B show plots of the temperature distribution the electric field distribution, respectively, after 90 seconds of freezing with an applied voltage of 2000V for 90 pulses.

FIGS. 15A and 15B show plots of the temperature distribution the electric field distribution, respectively, after 90 seconds of thawing with an applied voltage of 2000V for 90 pulses.

FIGS. 16A and 16B show graphs of the temperature distribution and electric field distribution, respectively, for the test case of FIG. 12A during 90 seconds of freezing.

FIGS. 16C and 16D show surface plots of the temperature distribution and electric field distribution, respectively, for the test case of FIG. 12A during 90 seconds of freezing.

FIGS. 17A and 17B show plots of the temperature distribution and electric field distribution, respectively, after 90 seconds of freezing with an applied voltage of 400V for 90 pulses.

FIGS. 18A and 18B show graphs of the temperature distribution and electric field distribution, respectively, for the test case of FIG. 12A during 90 seconds of thawing.

FIGS. 18C and 18D show surface plots of the temperature distribution and electric field distribution, respectively, for the test case of FIG. 12A during 90 seconds of thawing.

FIGS. 19A and 19B show plots of the temperature distribution and electric field distribution, respectively, after 90 seconds of thawing with an applied voltage of 400V for 90 pulses.

FIGS. 20A, 20B and 20C show the electric field distribution for the test case of FIG. 12A for the control case (FIG. 20A), freezing (FIG. 20B) and thawing (FIG. 20C).

FIGS. 20D, 20E and 20F show surface plots of the electric field distribution for the test case of FIG. 12A for the control case (FIG. 20D), freezing (FIG. 20E) and thawing (FIG. 20F).

FIGS. 21A and 21B show graphs of the temperature and electric field distribution, respectively, for the test case of FIG. 12 B during 90 seconds of freezing.

FIGS. 21C and 21D show surface plots of the temperature and electric field distribution, respectively, for the test case of FIG. 12 B during 90 seconds of freezing.

FIGS. 22A and 22B show plots of the temperature distribution and electric field distribution, respectively, after 90 seconds of freezing with an applied voltage of 400V for 90 pulses.

FIGS. 23A and 23B show graphs of the temperature and electric field distribution, respectively, for the test case of FIG. 12 B during 90 seconds of thawing.

FIGS. 23C and 23D show surface plots of the temperature and electric field distribution, respectively, for the test case of FIG. 12 B during 90 seconds of thawing.

FIGS. 24A, 24B and 24C show the electric field distribution for the test case of FIG. 12B for the control case (FIG. 24A), freezing (FIG. 24B) and thawing (FIG. 24C).

FIGS. 24D, 24E and 24F show surface plots of the electric field distribution for the test case of FIG. 12B for the control case (FIG. 24D), freezing (FIG. 24E) and thawing (FIG. 24F).

FIG. 25 is a schematic diagram of the positioning of the cooling probe and electrodes for a parallel circuit test setup of different variations.

FIG. 26 illustrates a finite element mesh utilized for the case in which two typical electroporation probes are used to deliver the electric pulses and one cold probe is used to cool the tissue and induce local changes in electric properties.

FIGS. 27A-D show plots of the electric field distributions along a transection at the diameter of the test setup of FIG. 25 for control (FIG. 27A) cold probe 4 cm from center (FIG. 27B), cold probe 2 cm from center (FIG. 27C), and cold probe 0 cm from center (FIG. 27D).

FIG. 28A-C show the area of tissue that undergoes irreversible electroporation for the control case without any cooling applied (FIG. 28A), cooling probe located 2 cm from the center (FIG. 28B), and cooling probe located at the center of the tissue (FIG. 28C).

FIGS. 29A and 29B show side and top schematic views, respectively, of a test setup performed on liver tissue.

FIGS. 30A and 30B show comparisons of the histology of the cryo-only treated tissue (FIG. 30A) and tissue treated with the device shown in FIGS. 29A-B (FIG. 30B).

### DETAILED DESCRIPTION OF THE INVENTION

The combined use of freezing and electroporation including electric fields (EF) is intertwined through the effect of freezing and temperature on the electrical properties of tissue. The systems and methods of the present invention exploit the point of intersection between freezing and electric fields to provide useful and novel applications of the combination of freezing and electric fields that cannot be accomplished by each one separately.

The terms “cryo” and “cryogenic” are sometimes understood in the art as pertaining to temperatures below −160° C. However, for purposes of this description, the terms “cryo” and “cryogenic” are herein defined according to their broader meaning in the biological arts. In particular the terms “cryo” and “cryogenic” are herein defined as of or pertaining to the production or affects of low temperatures.

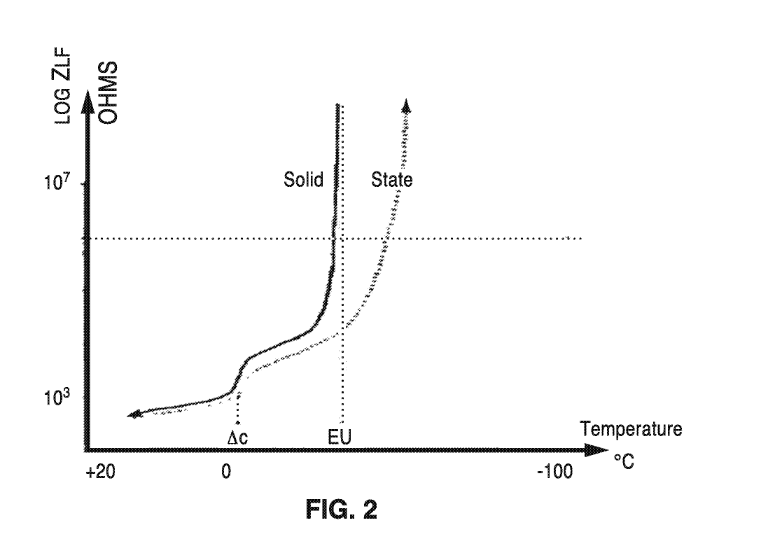

FIG. 2 shows the experimentally determined impedance of a physiological saline solution on a log scale in ohms, as a function of temperature during freezing (dashed line) and thawing (solid line). The eutectic (EU) point is −21.2° C. and the phase transition temperature of physiological saline is −0.56° C. It is important to note that the impedance increases with a decrease in temperature. The increase is particularly pronounced with the onset of freezing and it reaches extremely high values above the eutectic −21.2° C.

The systems and methods of the present invention utilize the combined effects of cold and freezing with electric fields to beneficially alter the electric circuit that forms in biological matter during application of these fields: The application of freezing and cold change the electrical properties of the tissue, which in turn modifies the electric fields in the tissue.

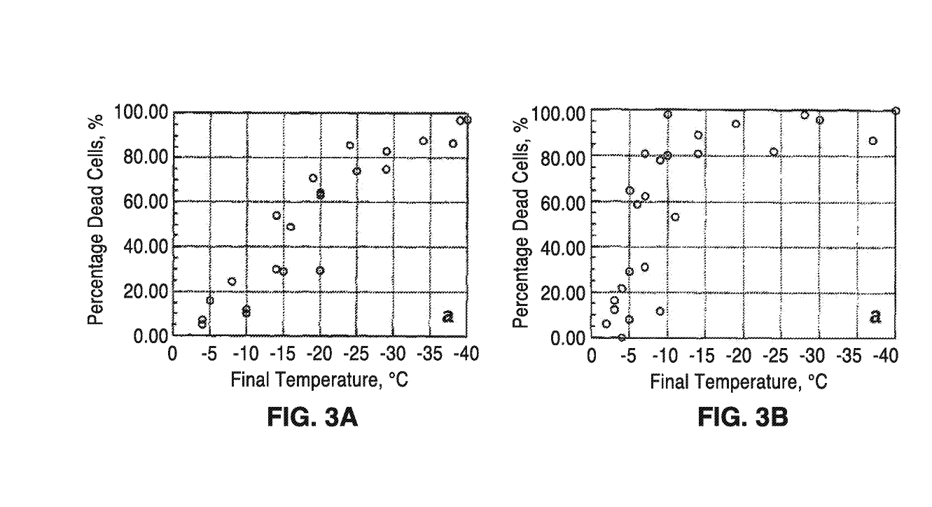

Two important parameters that affect the survival of frozen cells are the temperature to which they are frozen and the cooling rate during freezing. This is illustrated in FIGS. 3A and 3B, which show the effect of freezing temperature and cooling rate during freezing on cell death in ND-1 prostate cancer cells, and the percentage of dead cells as a function of the temperature to which they are frozen for various cooling rates during freezing. FIG. 3A plots cells frozen with a cooling rate of 5° C./min, and FIG. 3B shows cells frozen with a cooling rate of 25° C./min. It is important to note that cells survive freezing at temperatures below −5° C. for both cooling rates and that increasing the cooling rates during freezing will increase cell death. In this invention we will take advantage of this paradoxical effect in new ways, through combination with the effect of electric fields on tissue.

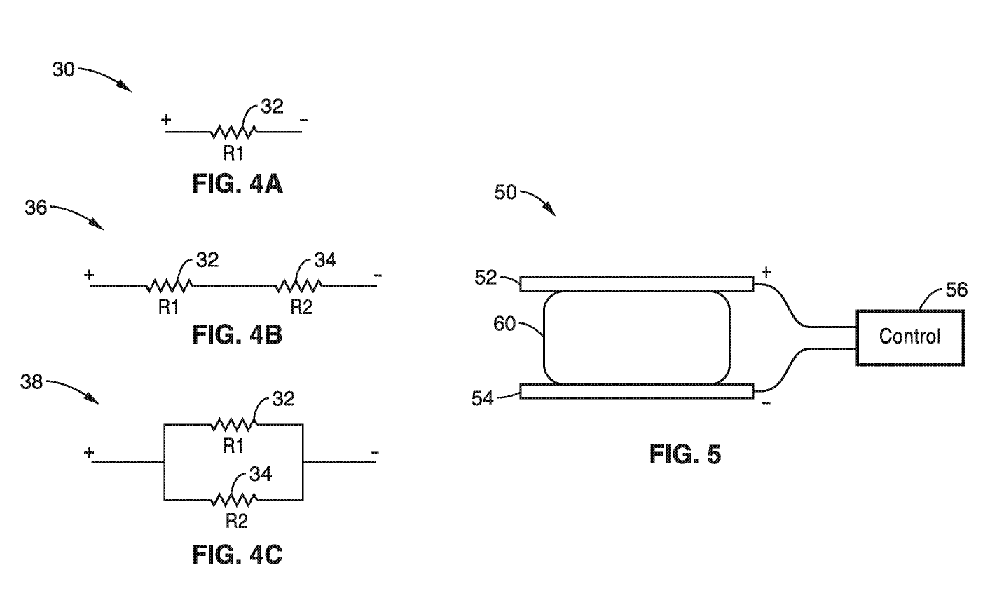

FIGS. 4A through 4C illustrate schematic diagrams of three configurations of the present invention for treating biological matter through a combination of delivery of electric fields and cold/freezing.

Configuration 30 shown in FIG. 4A illustrates an embodiment in which the entire tissue 32 is frozen and the electric potential is applied across the frozen region. The important effect in this case is the increase in resistance R1 (of the frozen tissue) with lower temperatures. The primary effect in this configuration will be a decrease in current with a decrease in temperature and with freezing. As shown in FIG. 3, at temperatures lower than eutectic, the impedance is very high and conventional understanding is that no DC current can flow through this configuration.

Configuration 36 shown in FIG. 4B illustrates an embodiment in which a part of the tissue 32 is frozen and part is unfrozen 34, with one electrode (e.g. positive) residing in the frozen domain or tissue and the second electrode (e.g. negative) in the unfrozen domain or tissue, acting a circuit in series. The electrical impedance R1 of the frozen lesion 32 is substantially higher than that of the unfrozen lesion 34. When an electric potential is applied between the two electrodes in this configuration, most of the potential drop occurs across the higher resistance (frozen lesion 32), which will therefore experience a much higher electric field than the physiological temperature tissue 34. The effect of this configuration is to increase the electric field across the frozen lesion 32 and decrease the electric field across the physiological temperature region 34, thereby focusing the delivery of the electroporation including EF on the frozen tissue 32. Configuration 36 has the beneficial result of affecting with EF a well defined part of tissue, which can be delineated by first freezing or cooling that part of the tissue and employing the EF in the configuration of mode 36.

Configuration 38 shown in FIG. 4C illustrates a third embodiment in which a first part 32 of the tissue is frozen and a second part 34 is unfrozen, however the positive and negative electrodes are both in a tissue with the same thermodynamic state, acting as a circuit in parallel. For instance, configuration 38 may be configured such that the positive and negative electrodes are in the physiological temperature region and a cryoprobe is freezing a volume of tissue in which it is desired to avoid the effects of the EF. In this circumstance, the current will be flowing primarily through the low resistance (R2) path of unfrozen tissue 34, and the current going through the high impedance (R1) frozen lesion 32 will be negligible. When freezing is done at high subzero temperatures, the cells in that volume 32 will survive because in that volume the freezing is with such parameters that do not induce damage, while the EF are with such local parameters that also do not induce damage (e.g. shielded from EF).

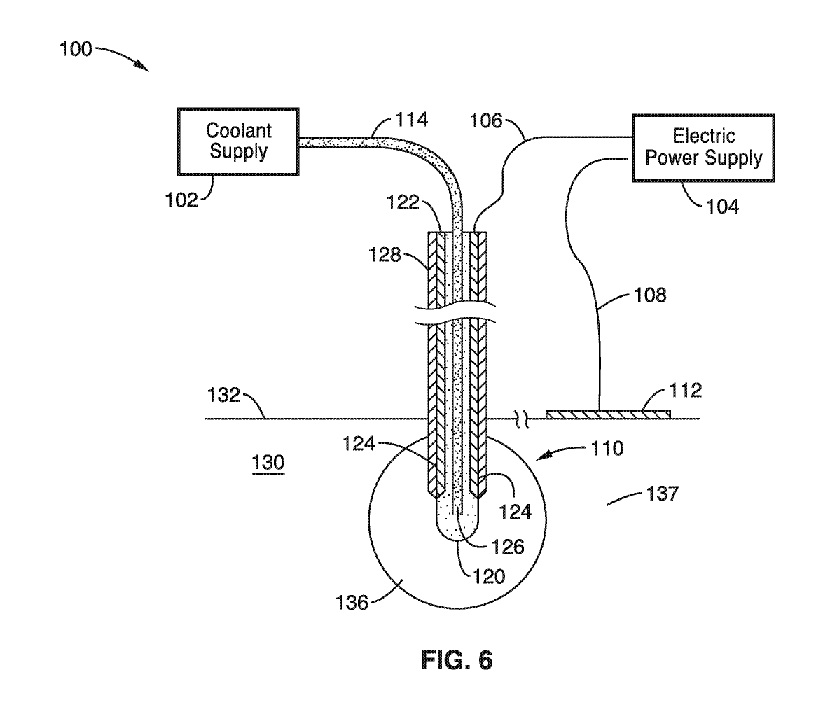

FIG. 5 shows a sterilization system 50 configured for applying EF to a uniformly frozen biological matter 60 for sterilization of the biological matter 60 similar to the configuration 30 of FIG. 4A. System 50 comprises a pair of electrode plates 52, 54 configured to be positioned in contact with opposing sides of the frozen biological matter 60. A controller 56 is coupled to the electrode plates 52, 54 for applying an electric field EF across the frozen biological matter 60. System 50 may be configured to be a standalone unit, or incorporated with a refrigeration/freezer system configured to freeze the biological matter or foodstuff.

An experiment was conducted to demonstrate that pulsed electric fields delivered to frozen cells, as provided in the system 50 of FIG. 5, can destroy frozen cells that survive freezing, particularly for the application to sterilization of frozen biological matter contaminated with microorganisms.

In this experiment, E. coli bacteria were plated on an LB Agar plate overnight. The following day one CFU was removed from the LB Agar plate and a 50 mL LB Broth+50 uL AMP was inoculated. Inoculated LB broth was placed in a shaking incubator overnight (37° C.) to reach stationary phase (12-14 hours). 100 ul of the LB broth with E. coli was removed and placed in 2.7 mL of sterile tap water (sample stored in the incubator when not in use).

In each experiment, the survival of E. coli was compared in: a) untreated controls, b) sample frozen to −5° C., c) samples frozen to −5° C. and electroporated with a typical electroporation sequence of 150 pulses of 15 kV/cm, and delivered for 50 microseconds each at a frequency of 1 Hz. The experiments were performed in 1 mm gap cuvettes (Electroporation Cuvette (Genesee Scientific)). Two BTX 830(Harvard Experiment) electroporation chambers were placed in a freezer and allowed to equilibrate to −17° C. A cuvette with tap water+E. coli (90 uL) was placed in BTX chamber 1 (located in the freezer) and used to assess temperature. A thermocouple was placed in the cuvette and the temperature was continuously monitored. A second cuvette (90 uL) was placed in the same BTX chamber and used as the sample that experienced only freezing. A third cuvette was placed in a second BTX chamber (also located in the freezer) and electroporated at (15 kV, 50 microseconds, 150 pulses, 1 Hz) when the temperature monitored by thermocouples in the water with E. coli cuvette reached −5° C.

Electroporation occurred between −4.9° C. to −5.9° C. It took approximately 2 min to reach −5° C. from 0° C. After electroporation, all samples were removed from the freezer and allowed to warm up to room temperature. Nine repeats of the experiment were performed. All samples were placed in the incubator and cell viability was evaluated with an Invitrogen BAC Light Live Dead Assay. A ten microliter per sample was removed from each individual cuvette and stained with the prepared BAC Light Assay. Samples were allowed to sit for 15 minutes and then transferred to the Flow Cytometry facility. Analysis of the microorganism viability was performed. It was found that in samples frozen without PEF to −5° C., the survival was 58%+/−25% of the original microorganism number of cells. When samples were exposed to PEF pulses in a frozen state, the percentage survival was substantially lower, only 8%+/−2.5%. This demonstrates that pulsed electric fields are effective when delivered to cells in a frozen state. Furthermore, delivering sterilization electric fields during frozen storage can be effective in reducing the microorganism load.

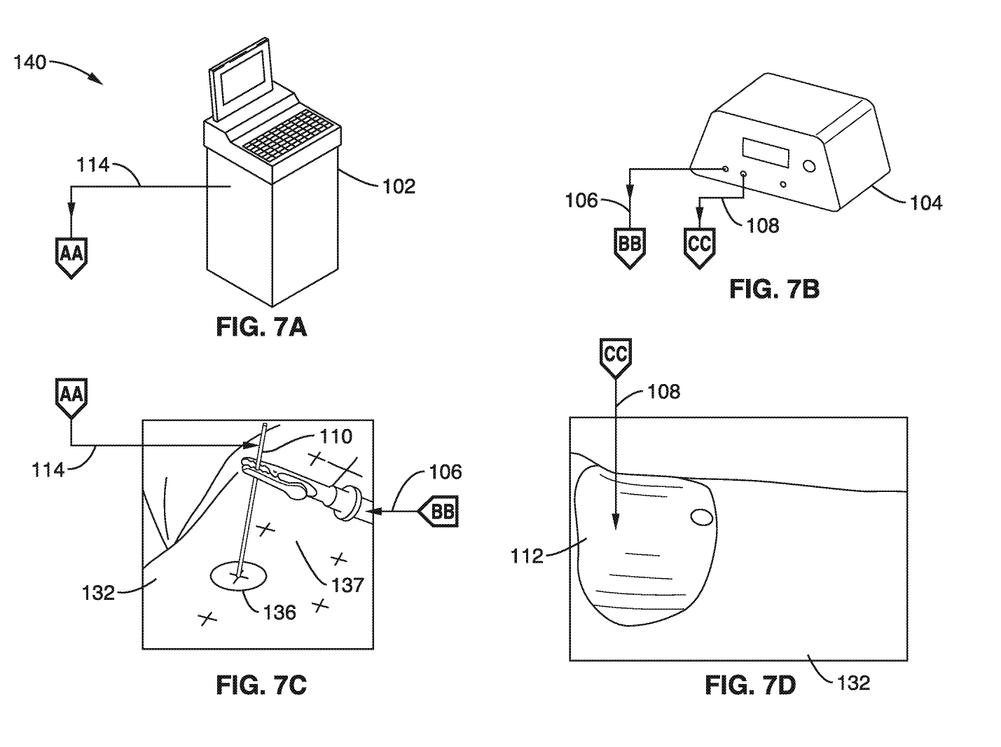

FIG. 6 illustrates a schematic diagram of a cryoelectric system 100 that is configured to operate in the mode 36 of FIG. 4B in accordance with the present invention. System 100 comprises of a source of coolant 102 and a source of electric power 104 both connected to a thermal probe 110. Probe 110 comprises an active portion 124 that is thermally and electrically conductive, and the passive part outer portion (e.g. sleeve) 128 is thermally and electrically insulated. The active portion 124 of the probe 110 is configured to conduct both thermally and electrically at distal end 120, which is configured to be in direct contact with the target tissue 130 to be treated.

The coolant source 102 is connected with conduit 114 for cooling means delivery to proximal end 122 of the probe 110 via a central channel 126 of the probe 110. It is appreciated that the supplied coolant may be any common refrigerant means, e.g. Freon or the like, that is capable of high freezing temperatures (e.g. between −30° C. and 0° C., and preferably from −5° C. and 0° C.), and not need to be liquid nitrogen or helium necessary for low temperature cryogenic applications.

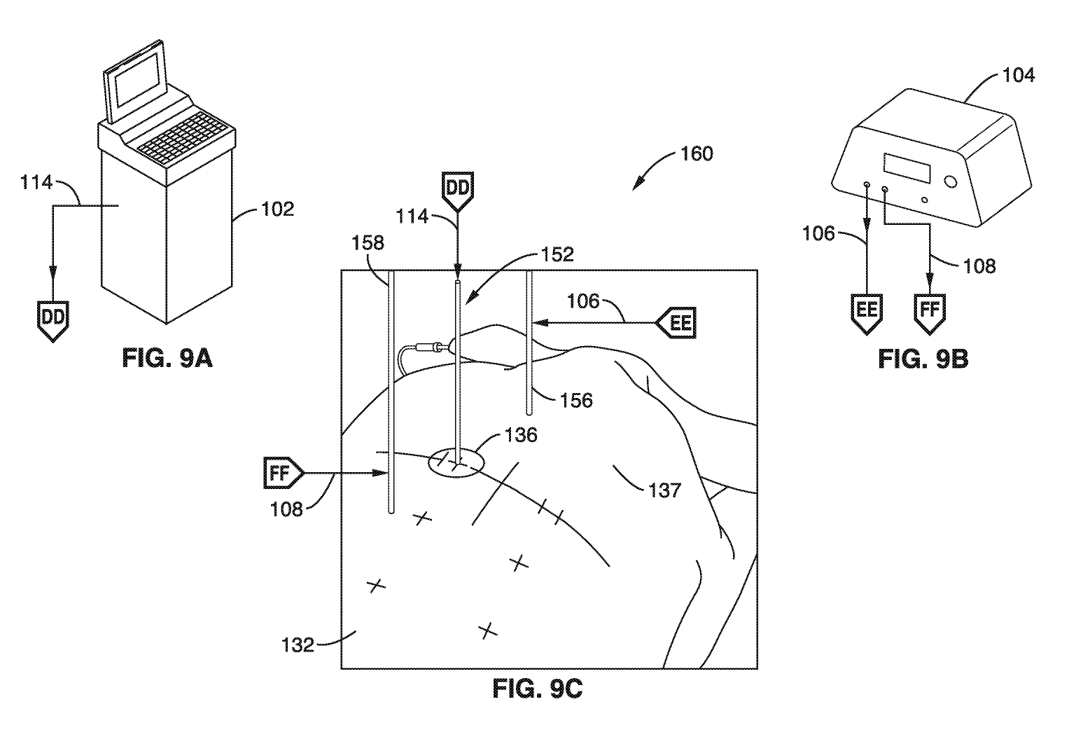

The electric power supply 104 is coupled to the conducting surface 124 of the probe 110 via a first lead 106. The electrical circuit in the tissue 130 is completed by connecting the second lead 108 from the electric power supply 104 to a conductive pad 112 (e.g. Rita pad) positioned on a remote part of the tissue surface 132 that is within non-frozen tissue 137 and outside the frozen region 136 generated from the cooling probe 110. Thus, the cooling probe 110 acts as a first electrode, while the pad 112 acts as a second or return electrode. It is also appreciated that the second electrode may comprise a probe (e.g. similar to probe 156 or 158 in FIG. 8) rather than a conducting pad.

The coolant supply 102 is configured to provide flow of coolant to the cooling or freezing probe 110. In one example, coolant supply 102 may comprise a conventional cryosurgery unit (e.g. Endocare Cryo 20). It should be emphasized that numerous different types of cooling systems may be used, and thus the system 100 is not limited to any one cryogen/refrigeration source.

In one exemplary embodiment, the electric power supply 104 comprises a BTX 830 electroporation system. It is also appreciated that numerous different types of electric power sources may be used, and thus the system 100 is not limited to any one specific power source.

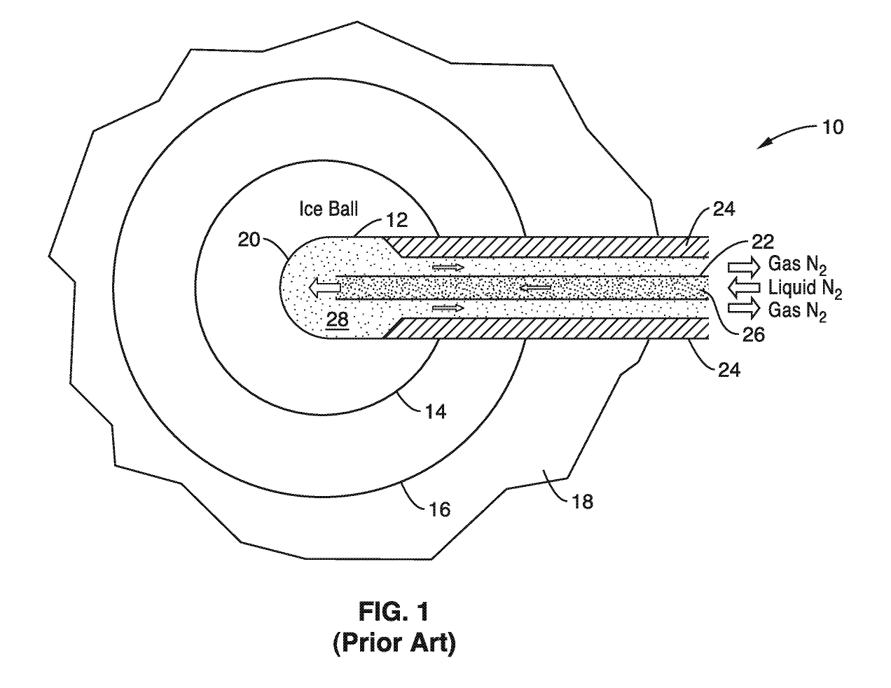

In one exemplary embodiment, the cryoelectric probe 110 may comprise an Endocare cryosurgery probe (e.g. the probe shown in FIG. 1) modified to have outer insulation layer 128, and connection means to connect to both the coolant supply 102 and the EF supply 104. As explained above for FIG. 1 the cryosurgery probe 10 is configured for delivering cryogenic (e.g. below −150° C.), and to the extent the probe 110 of the present invention only needs to deliver high freezing cooling (e.g. from −30° C. and 0° C.) and associated coolant (e.g. Freon, etc.), the configuration of probe 110 may vary accordingly.

The system 100 is configured to deliver EF and simultaneous cooling to the tissue 130 to generate a freezing zone or sphere (e.g. similar to isotherm 16 in FIG. 1) emanating from distal tip 120 of the probe in a series-type circuit scheme as shown in mode 36 of FIG. 4B. This creates an irreversible and isolated kill zone within the isotherm 136, while leaving tissue 130 outside the isotherm 136 free from cell destruction.

The system 100 is shown with coolant supply 102 and electric power supply 104 as separate sources. However, it is appreciated that the coolant supply 102 and electric power supply 104 may be integrated as one unit configured to both control and supply delivery of cooling means and EF to the tissue 130.

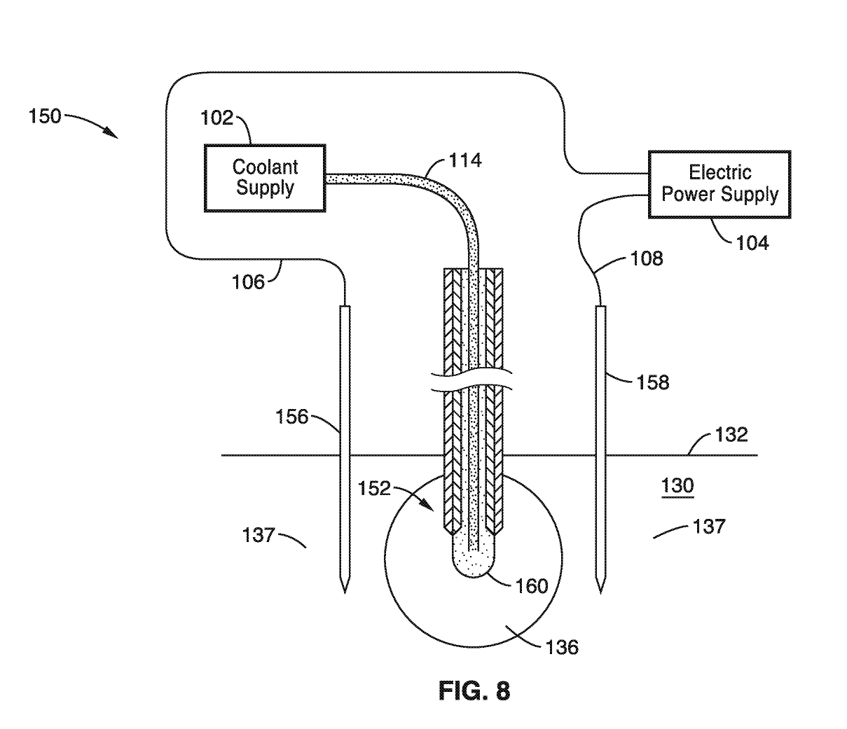

FIG. 7 shows a test setup 140 for the cryoelectric system 100 of FIG. 6. Test setup 140 is shown with the cryoelectric probe 110 inserted in the back muscle of a pig (e.g. tissue surface 132). The probe 110 is coupled to both external coolant supply 102 and EF controller and power supply 104 via conduit 114 and first lead 106. The electrical circuit is closed with second lead 108 and an electrode pad (Rita) 112 that are connected to the second outlet of the BTX power supply 104.

The configuration of setup 140 allows application of electric field pulses through the frozen lesion, which is shown as circular area 136 surrounding probe 110. The second electrode (pad) 112 rests on surface 132 in the unfrozen tissue 137. The configuration of FIG. 7 facilitates real time imaging of the tissue region 136 that is affected by both electric fields and freezing through imaging of the freezing process.

There are several alternative options for implementing the system 100 or test setup 140 of FIGS. 6 and 7 respectively. One configuration is to set the cryoprobe 110 to about −5° C. to −10° C. while continuously (and simultaneously) applying the EF from source 104 during the freezing. This will ensure that the frozen lesion is ablated by the EF.

A second configuration is to freeze to cryogenic temperatures and then let the tissue 130 thaw. The thawing process of freezing lesions is of such a nature that first the temperature of the frozen region increases towards the phase transition temperature and then stays at this high subzero temperature throughout the thawing process. This produces a large volume of frozen tissue at high subzero temperatures. The EF can be subsequently applied from the cryoelectric probe 110 throughout the thawing process to ensure the ablation of the entire frozen lesion. EF can be also applied continuously throughout freezing and thawing to maximize the effect.

FIG. 8 illustrates a schematic diagram of a shielding cryosurgery system 150 in accordance with the present invention, which generates a parallel electrical circuit with the tissue 130 similar to mode 38 of FIG. 4C. In system 150, first and second electrodes 156, 158 are connected to an EF power supply 104 via leads 106 and 108, respectively. A cooling probe or “cryoprobe” 152 is connected to a cooling source 102 and disposed in tissue 130 between first and second electrodes 156, 158. It is also appreciated that one or both of the first and second electrodes 156, 158 may comprise a pad (e.g. similar to pad 112 in FIG. 6) rather than a probe.

First and second electrodes 156, 158 are spaced a distance from probe 152 in a remote part of the tissue surface 132 that is within non-frozen tissue 137 and outside the frozen region 136 generated from the distal end 160 of cooling probe 152. Because the positive and negative electrodes 156, 158 are both in a tissue 137 with the same thermodynamic state, the system 150 acts as a circuit in parallel. Thus, current will be flowing primarily through the low resistance path of unfrozen tissue 137, and the current going through the high impedance frozen lesion 136 will be negligible. When freezing is done at high subzero temperatures, the cells in the volume 136 will survive because that volume is subjected to freezing parameters that do not induce damage, and the EF are within low local parameters that also do not induce damage. Thus, the frozen volume 136 is shielded from the potential damaging effects of EF within the surrounding tissue.

This can have particular benefit in treating the prostate, wherein the urethra (not shown) is positioned within the shielded frozen volume centered about cooling probe 152, while the prostate is being treated by EF pulses.

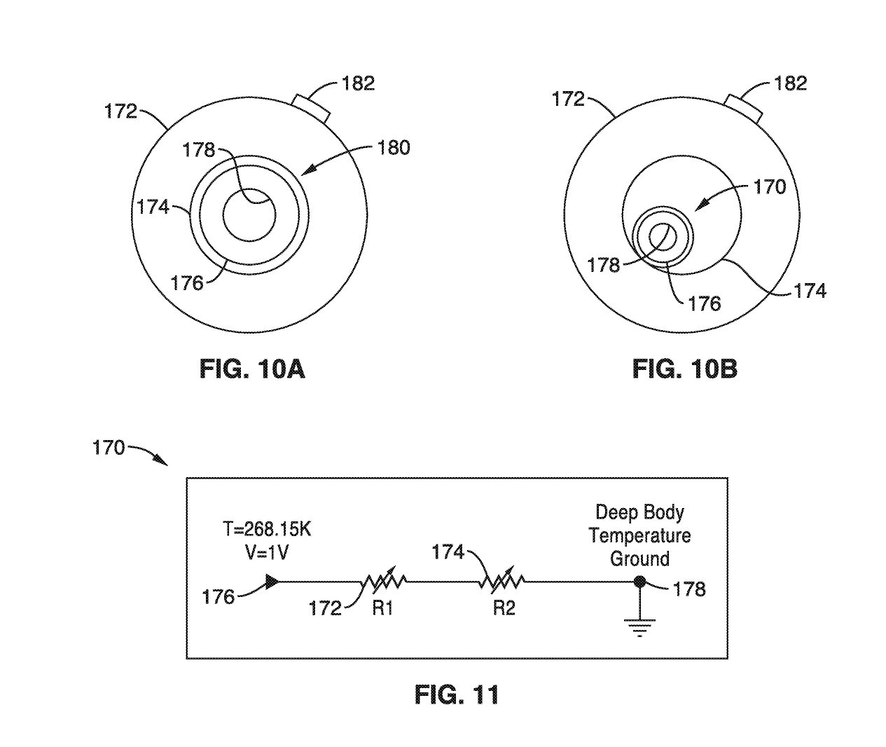

FIG. 9 shows a test setup 160 for the cryoelectric system 150 of FIG. 8. Test setup 160 is shown with the thermal or freezing probe 152 inserted in the back muscle of a pig (e.g. tissue surface 132). The thermal probe 152 is coupled to external coolant supply 102, while spaced apart electrodes 156 and 158 are positioned in tissue 137 that is outside the frozen thermal region 136 generated by freezing probe 152. EF controller and power supply 104 is coupled to electrodes 156 and 158 via leads 106 and 108. The freezing probe 152 is coupled to refrigerant source 102 via conduit 114.

The configuration of setup 160 allows application of electric field pulses within tissue 137 surrounding the shielded frozen lesion, which is shown as faint white circular area 136 surrounding probe 152.

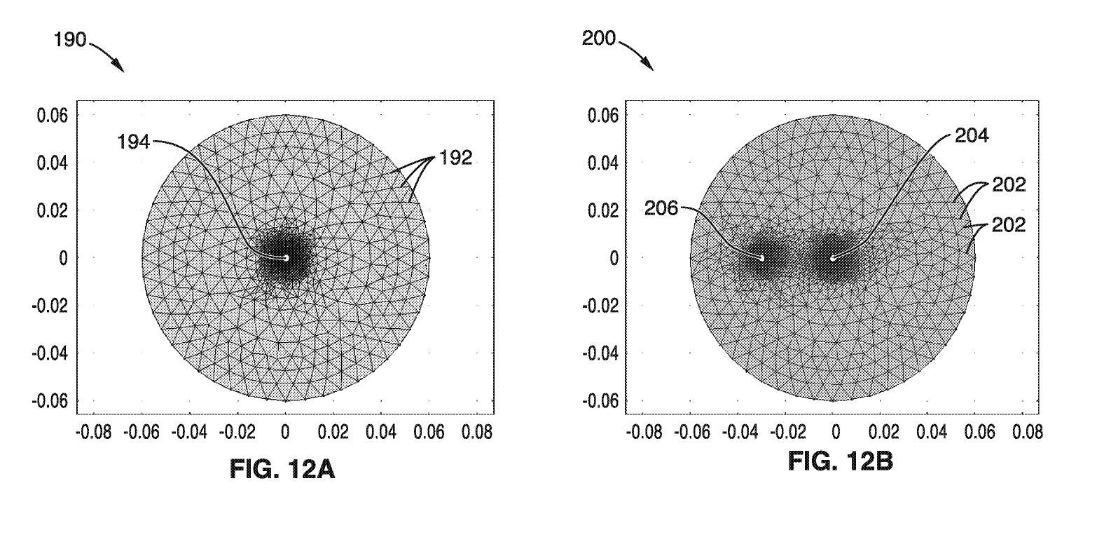

FIGS. 10A and 10B show schematic diagrams illustrating freezing through a cryoelectric probes 170 and 180 applied through a stick modality. In FIG. 10A, probe 180 is shown sized to have an outside diameter equal to or slightly smaller than the inner diameter of the inside wall 174 of the lumen 172 to be treated. In FIG. 10B, probe 170 is shown sized to have an outside diameter much smaller than the inner diameter of the inside wall 174 of the lumen 172 to be treated. It is appreciated that the same principles may apply to external or interstitial placement of the probes 170, 180 within or at the treatment location, and that the probes 170 and 180 may comprise different shapes (e.g. planar treatment surface, or the like).

PEF's are often used for applications in tissue cavities and surfaces, for instance inside blood vessels, the esophagus, colon, heart and on the skin. In these applications, it is beneficial or necessary to have good electric contact, which is normally achieved either with ballooning when done from the interior of the cavity or some mode of clamping to the surface when done on the exterior.

In a preferred embodiment, cryoelectric probes 170 and 180 comprise an internal lumen 178 for delivering refrigerant to the treatment tissue, and may include a similar system and setup to the probe 110 shown in FIG. 6. In such configuration, the cryoelectric probes 170 and 180 are coupled via a first lead to a power supply 104 (to form a first electrode), and the circuit is completed via second electrode 182, which may be positioned to contact an external surface of lumen 172, or another point in or in contact with the patient's body.

The stick properties of freezing when used with cryoelectric probes 170 or 180 generate excellent contact between the probe and the surface 14 via frozen layer 176, as illustrated for the interior of a cavity in FIGS. 10A and 10B. Therefore, freezing to high subzero temperatures will both focus the electric fields in the vicinity of the frozen lesion (e.g. layer 176) and ensure that the cryoelectric probes 170, 180 stick to the tissue 172 of interest without the need for additional modalities to establish the contact.

In one embodiment of the present invention, existing cryosurgical probes and devices can be modified for a cryoelectric application. The shaft of any cryoprobe is typically metal and electrically conductive, and therefore could also be used as an electrode. In fact, many cryosurgical probes also have an electrical conduit through the shaft, housing a thermocouple for the purpose of measuring temperatures at the tip of the cryoprobe. This could be one possible path for the electric pulses. Another possible path could be through direct connection to the metal shaft. To deliver the electric pulse only at the thermally active tip of the cryoprobe, it would be sufficient to apply a thin layer of electric insulation along the cryoprobe shaft, as in typical electroporation needles. The pulsed electric field power supply can either be stand alone, or incorporated in the cryosurgery console and connected to the electrically conductive cryoprobe shaft.

### Mathematical and Experimental Results

A. Mathematical Models

The cryoelectric tissue treatment systems and methods of the present invention were investigated utilizing a numerical mathematical analysis of temperature and electric fields produced by the application of EF together with freezing to verify and quantify the effect of changes in temperature and freezing on electric fields and the subsequent implications for treatment of tissues with the combination.

The models were generated using numerical analysis executed by Comsol Multiphysics (version 4.1). To extract the most salient biophysical aspects of the analysis, one and two-dimensional configurations were investigated in Cartesian and cylindrical coordinates. Each case utilized a coupled thermal and electrical model to simultaneously determine temperature and potential distributions during the simultaneous application of EF and freezing. To this end, two equations were solved simultaneously in Comsol. One was the Laplace equation (Eq. 1) for potential distribution associated with an electric pulse:

−∇·d(σ∇−J^{e})=dQⱼ Eq. 1

where σ is electrical conductivity, V is voltage, J^{e} is external current density, d is thickness and Qⱼ is the current source. For all cases, the thickness was set to one.

Physiological saline was used as a first order simulation of biological tissue. The electrical conductivity for saline was derived analytically for subzero temperatures using composite theory and the thermodynamic phase diagram for saline. The equation for freezing point depression was used to calculate the volume of solution as a function of temperature. Externally acquired experimental data was curve fitted to calculate the electrical conductivity of the composite medium. The derived electrical conductivity for subzero temperatures was combined with experimental data for higher temperatures, resulting in the following piecewise function:

$$σ ( T ) = \{ \begin{matrix}\frac{4.556}{T - 273.15} \times exp ( \frac{T - 273.15}{4.99962} ) - \frac{4.5559}{T - 273.15} + 2.365 e - 8 , T \leq 272.59 \\ 0.03 ( T - 273.15 ) + 0.7 , T > 272 , 59\end{matrix} \qquad Eq . 2$$

Eq. 2 above describes the behavior of electrical conductivity in [S/m] as a function of temperature [K], both above and below freezing. The correlation coefficient of this equation relative to experimental data was tabulated to be r=0.99989.

In addition to electrical conductivity, electrical permittivity is also a function of temperature. Eq. 3 was utilized to take into account the temperature dependence of electrical permittivity:

ε(T)=10^{(1.94404-1.99×10}^{^{−3}}^{(T-273.15))} Eq. 3

where ε is electrical permittivity and T is temperature in degrees K. Eq. 3 is valid for low frequency permittivity, experienced by typical PEF pulse parameters, which are in the range of 0.1-20E-3 seconds.

The thermal models were generated using numerical analysis executed by COMSOL MULTIPHYSICS (version 4.0). The temperature distribution was obtained from the solution of a modified Pennes bioheat equation, which was solved simultaneously as the electrical potential equation. The general bioheat equation has the following form:

$$∇ \cdot ( k ∇ T ) + {ρ}_{b} {w}_{b} {c}_{b} ( {T}_{a} - T ) + {q}^{′′′} = ρ {c}_{p} \frac{\partial T}{\partial t} , \qquad Eq . 4$$

where k is thermal conductivity, T is temperature, w_{b} is blood perfusion, c_{b} is the heat capacity of blood, Tₐ is arterial temperature, ρ is the tissue density, cₚ is the tissue heat capacity and q″′=Qₘₑₜ+Qₑₓₜ·Qₘₑₜ is the metabolic heat generation and Qₑₓₜ=σ|∇φ|² is a term that accounts for Joule heating, where φ is the electrical potential calculated from Eq. 1 and σ is electrical conductivity of the tissue. In this study, it was assumed that there is blood flow and metabolism in the unfrozen tissue while the blood flow and metabolism in the frozen region was set to zero. The effect of the electric field-induced Joule heating on the temperature distribution was considered in both the frozen and unfrozen tissues. The values for biological tissue utilized in the Pennes bioheat equation are listed in Table 1 and the thermal properties for the heat condition equation used are listed in Table 2.

The enthalpy method was utilized to account for the effects of freezing and thawing during cryosurgery. A heat transfer with phase change model, without electrical parameters, was compared to benchmark problems of this kind and validated the approach and results of this analysis. The values utilized in the heat conduction equation for frozen and unfrozen regions are shown in Table 1. For consistency with the electric field analysis, properties for physiological saline solution were also used to model the thermal behavior of biological tissue. All three values for frozen media in Table 2 were defined at temperatures below freezing, when T<272.59. The models defined values for unfrozen media in Table 2 at temperatures above freezing, when T>273.59. The transition region between the frozen and unfrozen media was defined using a smoothed Heaviside function. Therefore, the Heaviside function represented a volume fraction of liquid in the frozen media. The term for specific heat was modified to account for latent heat of fusion in order to model the phase transition:

C_{mod}=ΣᵢCₚ+Dλ Eq. 5

where λ is the latent heat of fusion (333E3 J/kg),

$$D = \frac{dH}{{dT}_{trans}} ,$$

H is the Heaviside function and Tₜᵣₐₙₛ represents the phase transition temperature.

Studies on the effect of temperature on electroporation protocols have revealed a negative correlation between temperature and fields. The electric fields required for producing electroporation increase as temperature decreases. The goal of this study is to investigate the ultimate effects of temperature modulation on PEF protocols, such as the fields necessary to induce reversible and irreversible electroporation. To accomplish this, data has been extracted from existing studies to produce a correlation between temperature and the electric fields at transition values between reversible and irreversible electroporation. The equation used in this study was derived from experimental data and is given by Eq. 6:

$$E ( T ) = \frac{- 39}{3200} T + 63700.45 , \qquad Eq . 6$$

where T is temperature [° C.] and E is electric field [V/m]. This equation was used as an approximate correlation for this study. It was used primarily to demonstrate an accurate methodology, however more precise correlations may also be applied. It should be noted that that the hyperosmolarity of the extracellular solution in the high subzero temperature range is expected to reduce the field required for electroporation. Therefore, Eq. 6 is anticipated to be an upper limit of the electric field required for electroporation at the conditions on the outer rim of the frozen lesion during cryosurgery.

B. Test Setup

The study employed three geometries that involve the use of a cryoelectric probe: a) Case 1, a one dimensional Cartesian geometry; b) Case 2, a one dimensional cylindrical geometry with a single cryoelectric probe in the center and an electrode at infinity (as in systems 100, 140 shown in FIGS. 6 and 7; and c) Case 3, a two dimensional cylindrical geometry with two cryoelectric probes. In all cases the analysis is performed prior to freezing (control at physiological temperature, 310.15K), during freezing, and during thawing. In the freezing case, a freezing temperature of 268.15K was applied to the cryoprobe. A temperature of 268.15K (−5° C.), was implemented because in very conservative estimates cell survival occurs at temperatures above 258.15K in cryosurgery. This range enables a test of the conditions in which PEF can ablate cells in a frozen lesion where frozen cells survive. Furthermore, this is a subzero temperature at which the experiments discussed earlier show that electroporation occurs. The duration of freezing was 90 seconds, after which the cold surface is thermally insulated and natural thawing was induced by constant deep body physiological temperature. The duration of the analyzed thawing period was also 90 seconds. While these periods of time for freezing and thawing are short relative to conventional cryosurgery, they are relevant to the relatively high subfreezing range of temperatures, which is the focus of this analysis. A voltage difference of 1V was used in the electric field analysis to facilitate a general normalized analysis of the electric fields.

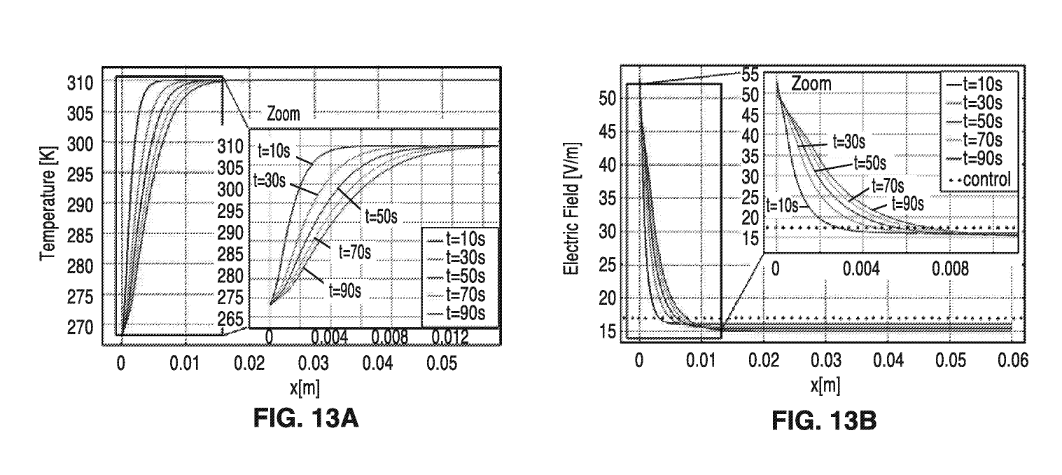

Case 1: Referring now to FIG. 11, the first study was performed using test setup 170 comprising of a simple one dimensional 6 cm slab of tissue between first and second parallel plates 176, 178 in Cartesian coordinates. This study was used to demonstrate the fundamental aspects of the cryosurgery/PEF in accordance with the present invention. Two resistors in series, representing the frozen 172 and unfrozen 174 portions of the tissue, characterize the electrical configuration of this problem. This was done because at sub-MHz frequencies, such as in this PEF analysis, capacitance can be neglected. The second plate 178 is assumed to be at constant deep body temperature, and the first plate 176 (the freezing (cryoelectric probe) plate), was set to 268.15K. The duration of freezing was 90 seconds, after which the cold surface is thermally insulated and natural thawing was induced by constant deep body physiological temperature. The duration of the analyzed thawing period was also 90 seconds. A voltage difference of 1V was imposed between the plates 176, 178 to facilitate a general normalized analysis of the electric fields.

An evenly distributed finite element mesh (not shown) was incorporated into the model. The mesh size was varied in order to validate the accuracy of the solution. The mesh size was refined until the solution was no longer affected by the quality of the mesh. The mesh for Case 1 consisted of roughly 1500 elements.

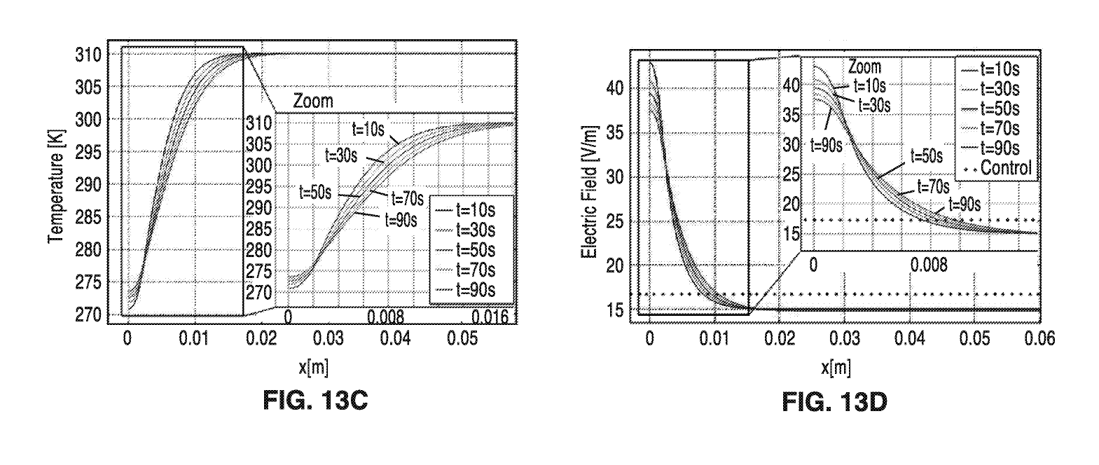

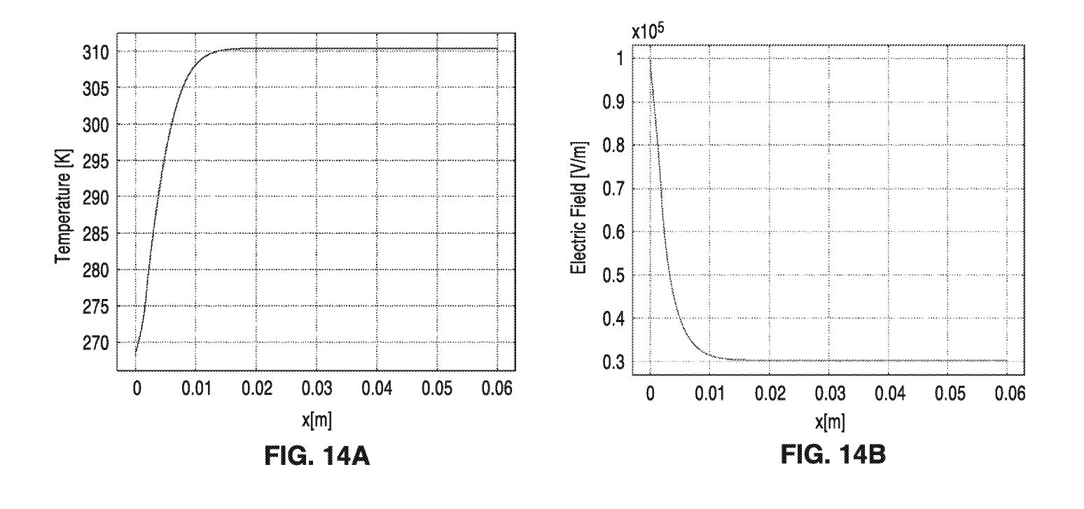

Case 2: The second geometry (FIG. 12A) comprised a cryoelectric probe 3.4 mm in diameter, inserted into the center of an infinitely long cylinder of tissue, 6 cm in radius. The outer edge of the cylinder was set to a constant deep body temperature and the temperature of the cryosurgical probe was set to 268.15K for 90 seconds of freezing. After freezing, the cryoprobe was modeled as thermally insulating to simulate tissue induced thawing for 90 seconds. The cryoprobe was modeled as thermally insulating when the flow of the cryogen is stopped because in a two dimensional configuration there is no axial heat flow and because typical cryoprobes are made of an insulated hollow thin walled tube, which gives them a negligible thermal mass relative to that of the frozen tissue and energy content in phase transition. The outer surface temperature of the tissue cylinder was maintained at a constant deep body temperature, which is what induces the thawing. A voltage difference of 1V was applied between the cryoelectric probe and the uniform outer edge of the tissue cylinder.

This geometry models a cryosurgical procedure during which a cryoelectric probe is used in tissue and a PEF voltage difference is applied between the cryoelectric probe and a ground electrode at a distance. The finite element mesh 190 utilized for Case 2 incorporated triangular elements 192, as shown in FIG. 12A. The element 192 size was smallest adjacent to the cryoprobe 194, and increased in size as it radiated towards the outer boundary. This was done in order to accurately capture the steep temperature gradient adjacent to the cryoprobe 194. The mesh was refined until the solution was no longer affected by mesh size. Approximately 3000 elements were utilized to cover a 113 mm² surface area.

Case 3: The third geometry (FIG. 12B) was an infinite cylinder of tissue 6 cm in radius represented in two dimensions. The outer margin of the cylinder was set to a constant deep body temperature and electrically insulated. Analyzed here is a simulation of a possible cryosurgery/PEF treatment protocol. In this simulation, one 3.4 mm cryoelectric probe 204 was inserted into the center of the tissue and a second 3.4 mm cryoelectric probe 206 was inserted 3 cm away. As shown in FIGS. 6 and 7, cryoprobes, which are generally made of a conductive material such as a metal, can be used as both cryosurgical probes when connected to the cryogen supply tank 102, and as electrodes when connected to a voltage supply 104.

In this simulation, the first cryoelectric probe 204 was set to 268.15K for 90 seconds of freezing. It was assumed that during this period the second cryoelectric probe 206 is not thermally activated, and therefore can be considered thermally insulated. Following the 90 seconds of freezing, the first cryosurgical probe 204 also ceased being thermally activated, and both probes became thermally insulating. This allowed the frozen tissue to thaw, which continued for 90 seconds. During both the freezing and thawing sequences described above, both cryoelectric probes 204, 206 were connected to the PEF power generation system and a voltage difference of 1V was imposed between them to facilitate a general normalized analysis of the electric field. The finite element mesh 200 in Case 3 utilized triangular elements 202, which are shown in FIG. 12B. The element 202 size was smallest in the region surrounding both probes 204, 206. This was done in order to accurately capture the temperature gradients adjacent to both of the probes. The mesh was refined until the solution was no longer affected by mesh size. Approximately 4000 elements were utilized to cover a 113 mm² surface area.

C. Results

Case 1:

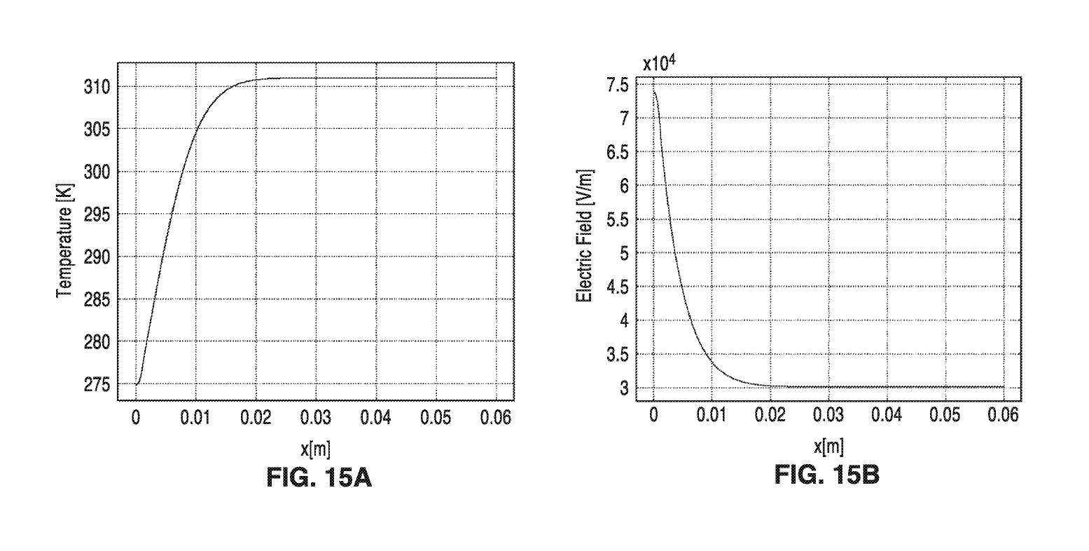

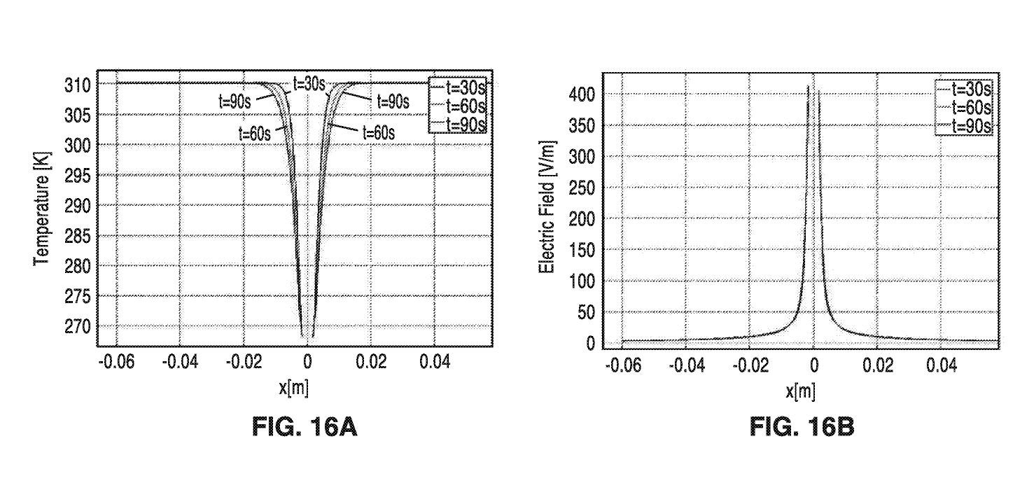

FIGS. 13A and 13B show graphs for temperature and electric field distribution, respectively, during 90 seconds of freezing.

Each line represents a 20 second time increment. The dotted line shows the electric field in tissue held constant at body temperature for the same voltage boundary conditions. The temperature distribution is as expected, increasing from the low temperature at the cryoprobe surface to body temperature. As time progresses, the low temperatures penetrate further into the tissue due to thermal diffusion. Note that at 272.59K, the nonlinear behavior indicates the region of phase change. FIGS. 13A and 13B demonstrate the inversely proportional relationship between temperature and electrical conductivity, as described by Eq. 3. Lower temperatures yield a lower ionic conductivity and subfreezing temperatures yield a dramatic decrease in electrical conductivity. From continuity of ionic current, the electric field will be higher in the regions of lower electrical conductivity. Indeed, FIGS. 13A-D illustrate the most important feature of the cryosurgery/PEF combination; because of the increased electrical resistance in the frozen and cooled regions of tissue, the highest electric fields are confined to those regions.

FIG. 13B also shows that the electric fields beyond the frozen and cooled regions in the normal tissue are substantially lower than those in the frozen/cooled regions. This suggests that the freezing/cooling has the effect of confining the high electric fields to those regions. The plots in FIGS. 13A-D were obtained for a normalized voltage of 1V. Combining FIGS. 13A-D and Eq. 6 suggests that a voltage of about 2000V on the cryoelectric probe is sufficient to ablate the analyzed frozen tissue, by PEF. Such voltages are typical to those used for tissue ablation with irreversible electroporation.

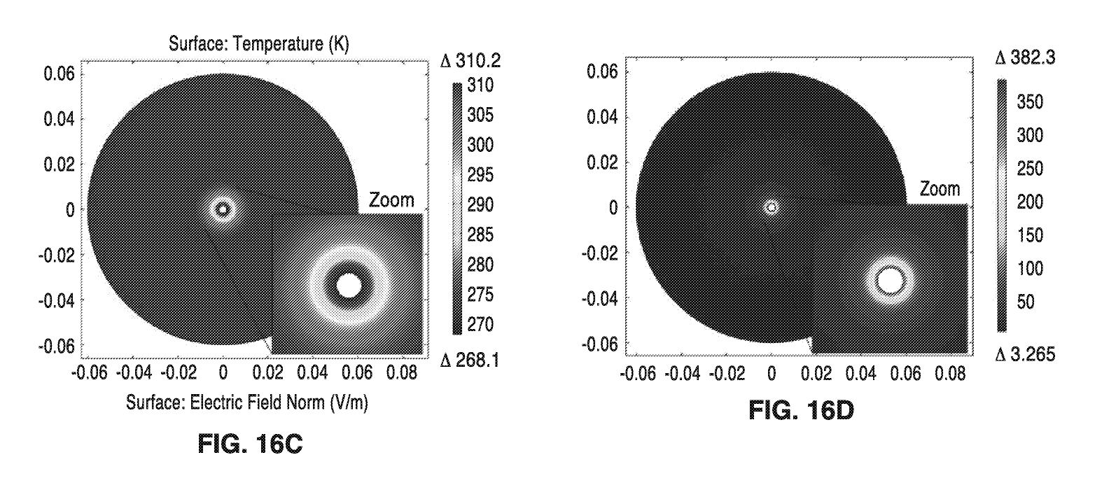

The result of applying 2000V after 90 seconds of freezing to the model in FIGS. 13A-B can be seen in FIGS. 14A and 14B. As can be seen by this graph, the temperature distribution in the tissue is similar to that in FIGS. 13A-B, where 1V was applied. However, the one difference is that temperature rises to a higher value over a shorter distance adjacent to the leftmost plate because Joule heating occurs at higher voltages. The electric field distribution in FIGS. 14A-B demonstrates the same trend as that of the 1V freezing model in FIGS. 13A-B. However, the maximum electric field reached is now 100000V/m. This results in an electric field distribution above 67000V/m, the threshold for irreversible electroporation, in the frozen region.

It should be emphasized that in this study the range of subfreezing temperatures used are relatively high, above −5° C. In this temperature range cells survive freezing. Adding PEF during freezing will ablate the cells in the frozen region that survive freezing, thereby making the cryosurgery/PEF treatment produce tissue ablation under conditions in which cells survive cryosurgery alone. This also suggests that the cryoelectric (cryosurgery/PEF) technique does not require the cryogenic temperatures used in conventional cryosurgery and cryosurgical systems. Therefore, cooling systems using Joule Thomson, solid state thermoelectric systems, or conventional refrigeration cycles may be sufficient for tissue ablation with this method. FIGS. 13A-B and this analysis show that the effect of freezing and low temperatures on electrical conductivity can actually concentrate the electric field to the cooled/frozen region, as well as amplify the electric field in that region, which would require substantial lower voltages on the cryoelectric probes than on conventional PEF probes.

FIGS. 13C and 13D show graphs for temperature and electric field distribution, respectively, during 90 seconds of thawing. FIG. 13C shows that adjacent to the cryoprobe, the temperature inches upwards, because no freezing temperature is applied by the probe during thawing. If thawing were extended beyond 90 seconds, the temperature graph would continue to equilibrate toward the phase transition temperature. The temperature distribution has a point of inversion, which appears to be stationary in time at about 0.2 cm from the outer surface. This point of inversion corresponds to the position of the change of phase interface.

The temperature history during thawing of frozen media has a peculiarity caused by the effects of the phase transition, which has been studied extensively in the past. This effect appears in FIG. 13C and is of significance to the use of PEFs during thawing. Previous studies have shown that during thawing of frozen lesions, the temperature of the frozen region increases first to the phase transition temperature, before the frozen tissue begins to thaw, and then remains at this value throughout the thawing process.

FIG. 13D shows that the relationship between temperature and electrical conductivity produces an electric field that also has a point of inversion around 0.2 cm, which is the outer edge of the frozen lesion. FIG. 13D also shows that the electric field in the frozen region drops precipitously during thawing to a constant value of 32V/m (for the 1V potential on the cryoprobe). FIG. 13D and Eq. 6 suggest that applying an electric potential above 2000V is sufficient to cause irreversible electroporation in the thawing region, in this example. Such voltages are typical to those used for tissue ablation with irreversible electroporation.

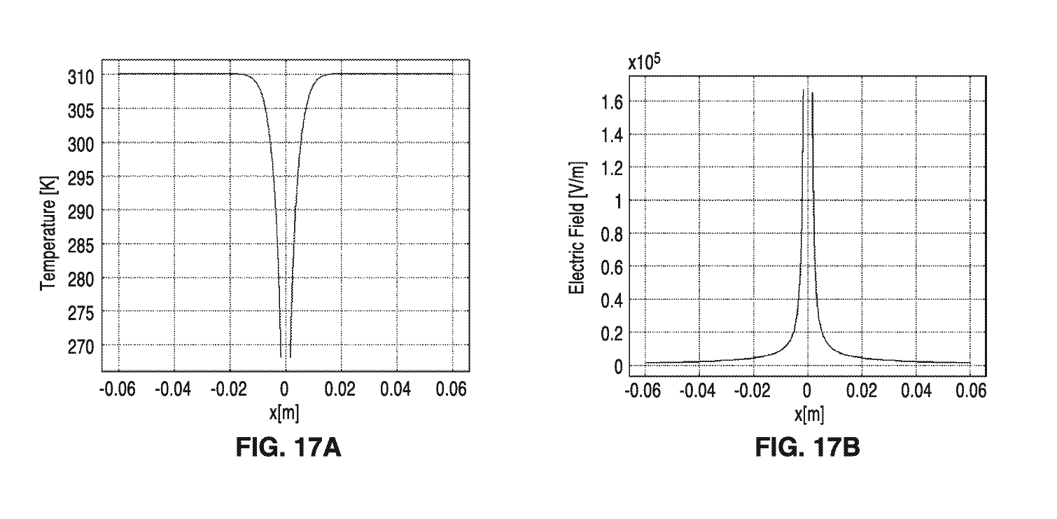

FIGS. 15A-B illustrates the temperature and electric field distributions, respectively, when 2000V is applied after 90 seconds of thawing. The temperature distribution is similar to that seen in FIG. 13C (when 1V is applied). However, the temperature rises to a higher value over a shorter distance when 2000V is applied due to Joule heating. The lowest temperature is at 275K in the 2000V case rather than 274K as in the 1V case. The electric field in the 2000V case has a similar distribution to the 1V case, except the highest electric field reached in this case is 75000V/m. It is apparent that if the temperature in the frozen region during thawing stays at the phase transition temperature, the electrical conductivity in the frozen region will remain constant throughout thawing and (in Cartesian coordinates) the electric field in the frozen region will remain constant as well. This suggests that if PEFs are applied during thawing they will be confined and delivered in the frozen region across cells that are all at the highest possible subfreezing temperature. This could allow for precise design of the PEF electrical parameters to take values that affect only cells in the thawing frozen tissue at the phase transition temperature.

The electric field produced during freezing was also compared to a control study. The control study applied the same electrical boundary conditions to tissue held at body temperature. The control study is represented by the dotted horizontal line in FIGS. 13B (freezing study) and 13D (thawing study). It is evident that the electric field in the frozen/cooled regions is substantially higher than the field produced in the control study in the same region in both freezing and thawing. However, at a distance from the frozen region, in the location of normal body temperatures, the fields are lower than those in the control. This suggests cryoelectric protocols of the present invention may be configured such that the PEF induced cell damage is confined to the frozen/cooled regions and the damage does not extend beyond the cooled regions.

Cryoelectric protocols may also be configured to induce cell damage via electrochemotherapy with reversible electroporation. It could be also used with reversible electroporation for the purpose of gene therapy and drug delivery. By combining cryosurgery with electrochemotherapy PEFs, the fields required for reversible electroporation in the liver are about 36,000 V/m. Therefore a pulse of about 700V will produce reversible electroporation in the region from −5° C. to +5° C. However, FIG. 13B shows that the same pulse will produce a field of approximately 10,000V/m in the body temperature region, which should have no effect on the tissue in that location. In contrast, in the control case (at constant body temperature), FIG. 13B shows that a pulse of about 1800V would be needed to produce the fields required for electrochemotherapy and the fields would affect the entire region between the electrodes.

Case 2:

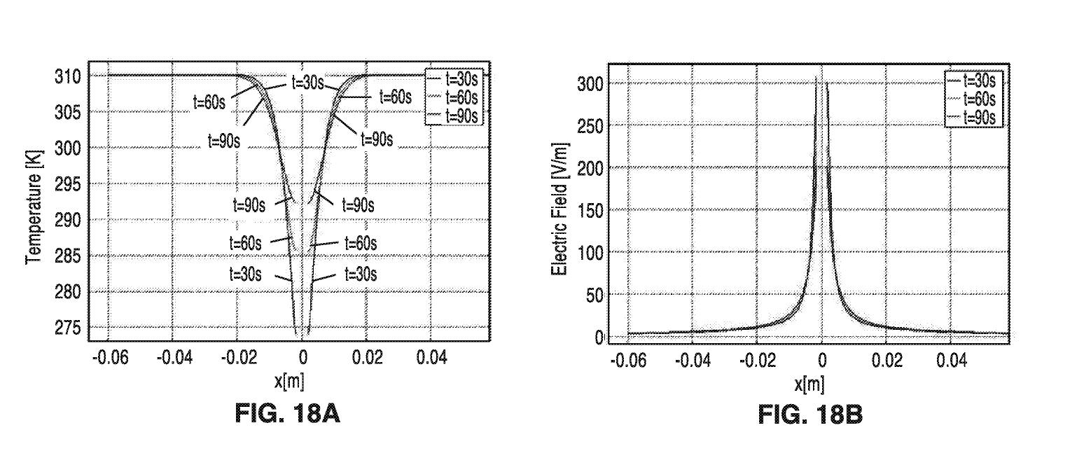

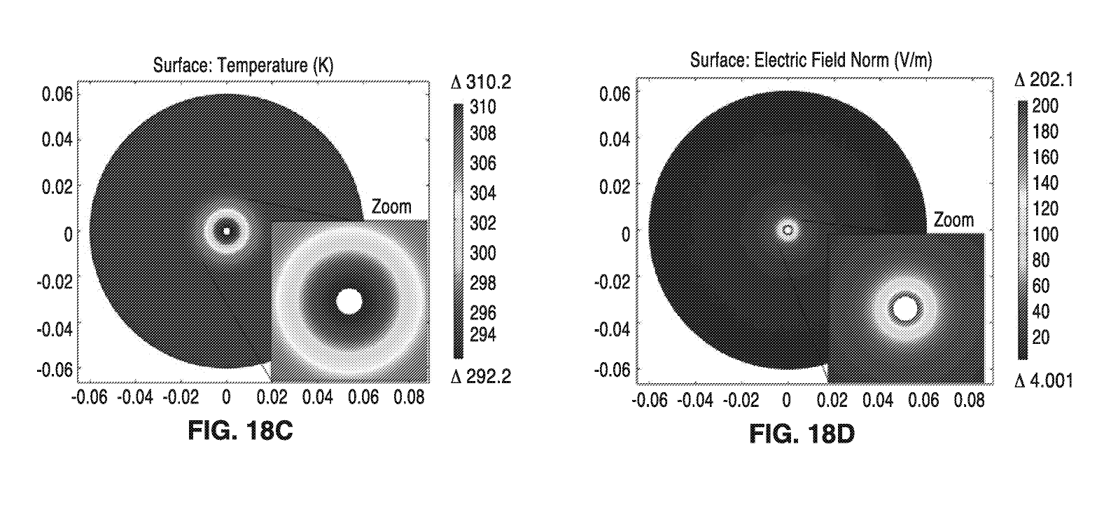

FIGS. 16A and 16B show graphs of the temperature distribution and electric field distribution, respectively, for the test case of FIG. 12A during 90 seconds of freezing. FIG. 16A illustrates the temperature distribution at a transection along the diameter. Note that in FIG. 16A, the gap in the plot is the location of the cryoelectric probe 194 (see FIG. 12A). As in the 1D Cartesian case, due to thermal diffusion, freezing temperatures penetrate further into the tissue with time. As well, nonlinear behavior at 272.59K indicates the region of phase change. The relationship between temperature and electric field is discernable from FIGS. 16A and 16B. As in the 1D Cartesian study, temperature and electric field are inversely proportional due to the dependence of electrical conductivity on temperature. Because of decreased electrical conductivity in the frozen and cooled tissue, the highest electric fields are confined to those regions.

FIG. 16B shows that the fields beyond the frozen and cooled regions are orders of magnitude lower than those in the frozen/cooled regions. This suggests that freezing/cooling temperatures have the effect of magnifying the high electric fields in those regions.

The effect of low temperature on confining the electric field is effectively demonstrated in FIGS. 16C and 16D. The zoom panel demonstrates that the electric field is confined within the cooled region. FIGS. 15 A-D show that the electric field in the frozen region is higher than about 150V/m for a voltage of 1V on the cryoprobe. FIGS. 15 A-D suggest that a voltage of about 400V imposed on the cryoelectric probe is sufficient to ablate the cells with PEF in the analyzed frozen region.

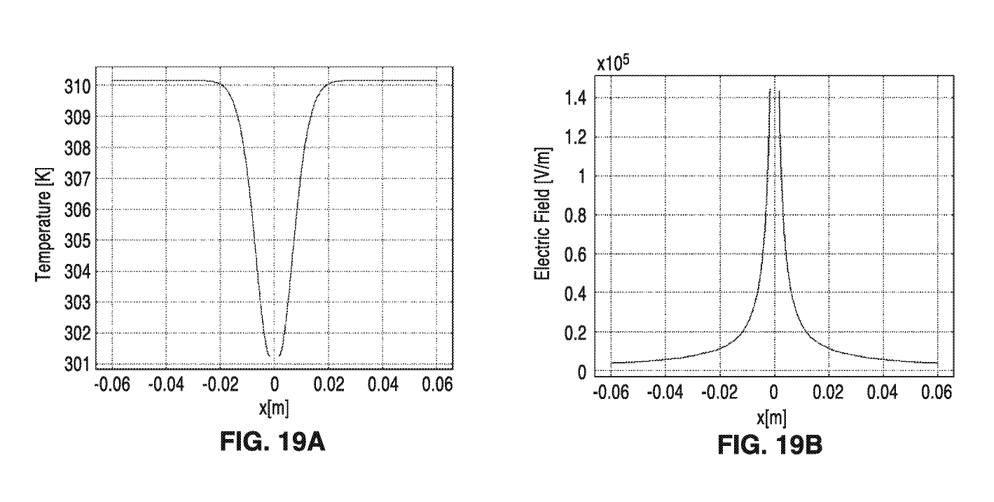

FIGS. 17A and 17B show plots of the temperature distribution and electric field distribution, respectively, after 90 seconds of freezing with an applied voltage of 400V for 90 pulses. When 400V are applied, the temperature rises to a higher value over a shorter distance than when 1V was applied (FIG. 16A) due to the effect of Joule heating. The electric field with 400V applied demonstrates the same trend as the case with 1V applied, but reaches a maximum of 160000V/m rather than the peak of 400V/m seen in the 1V case.

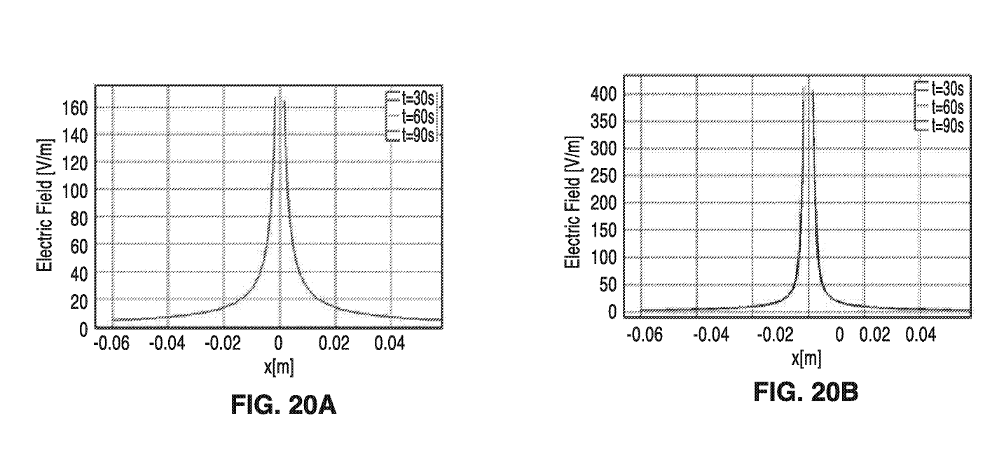

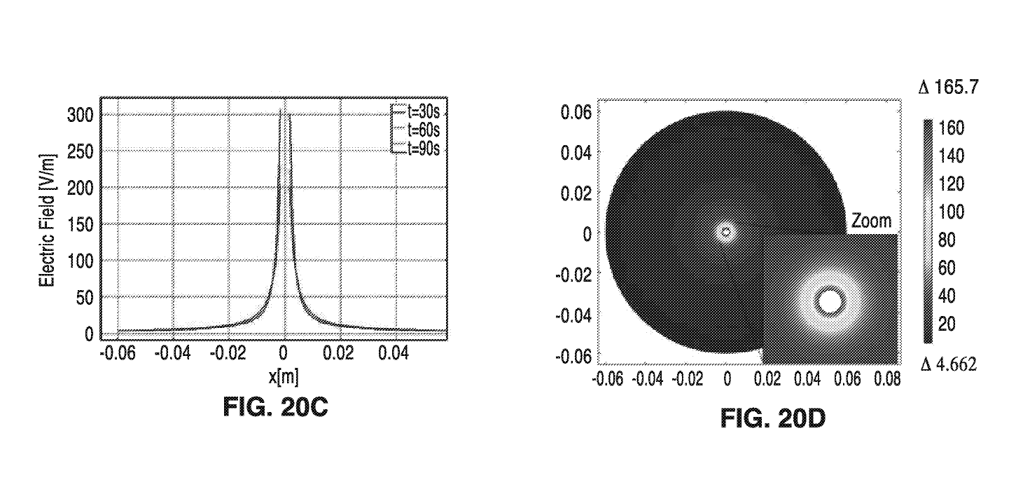

FIGS. 18A and 18B show graphs of the temperature distribution and electric field distribution, respectively, for the test case of FIG. 12A during 90 seconds of thawing. FIGS. 18C and 18D show surface plots of the temperature distribution and electric field distribution, respectively, for the test case of FIG. 12A during 90 seconds of thawing.

The temperature distribution during thawing with 1D cylindrical symmetry behaves very similarly to the previously discussed temperature distribution in the 1D Cartesian case. FIG. 18A shows that temperatures adjacent to the cryoelectric probe inch upwards as a result of its insulated boundary conditions. The temperature distribution has a stationary point of inversion at 0.6 cm. FIG. 18B shows that the electric field also has a point of inversion around 0.6 cm. FIG. 18B also demonstrates that the electric field in the frozen/cooled region during thawing drops dramatically, effectively confining the field. Because of the transient nature of the temperature distribution, the electric field also changes with time. For instance, 30 seconds into thawing the highest electric field near the cryoprobe has dropped from 400 V/m at the end of freezing to 300V/m.

The location of the highest electric field is the same point in space and time as the lowest temperature, which is consistent with Eq. 2. Additionally, the electric field decreases in time as thawing progresses and temperatures rise throughout the domain. The slope of the electric field also follows that of the temperature. The slope of the electric field is steepest at the onset of thawing, when temperatures are lowest. But as thawing progresses, and temperatures begin to rise, the slope of the electric field lessens. This indicates the strong relationship between electric field and temperature in the cryoelectric procedure and the ability of freezing/cold temperatures to confine the electric field.

This observation is most evident from FIGS. 18C and 18D, which depict surface plots of the temperature and electric field distribution, respectively, during the thawing stage of the cryosurgery/PEF procedure. The zoom panels clearly illustrate that the electric field is confined inside the lower temperature regions during thawing. FIGS. 18C and 18D also suggest that here also, similarly to the Cartesian one dimensional case, the field produced by 1V potential at the cryosurgery/PEF probe in the thawing frozen lesion will be higher than about 75V/m. Therefore, Eq. 6 suggests that a voltage of about 850V on the cryosurgery/PEF probe will be sufficient to ablate with PEF, the frozen cells during thawing.

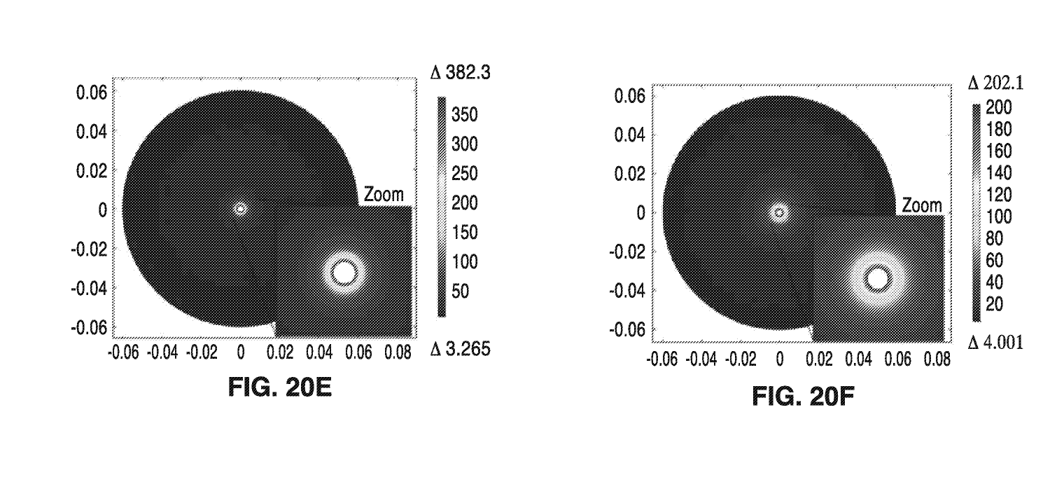

FIGS. 19A and 19B show plots of the temperature distribution and electric field distribution, respectively, after 90 seconds of thawing with an applied voltage of 850V. Due to the higher voltage, electric field induced Joule heating affects the temperature distribution. In the case of 850V, the lowest temperature in the domain is 301K, whereas with an applied voltage of 1V the lowest temperature is 293K. The electric field achieved due to 850V is also much higher. It reaches a peak of 140000V/m, whereas the peak in the 1V case is 300V/m.

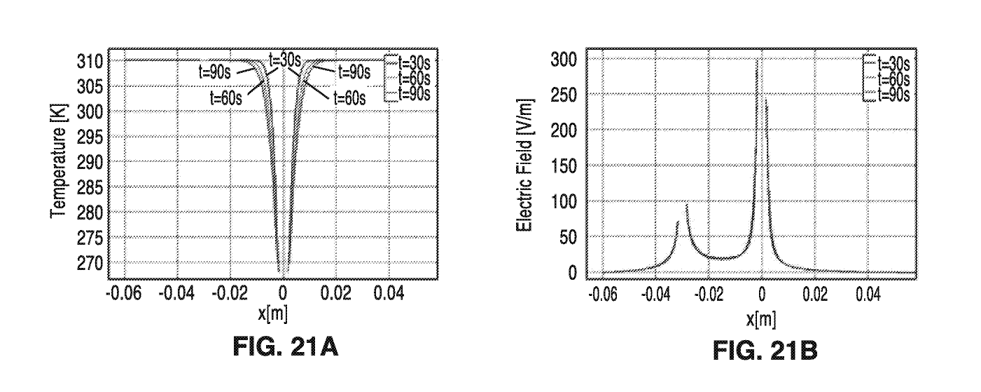

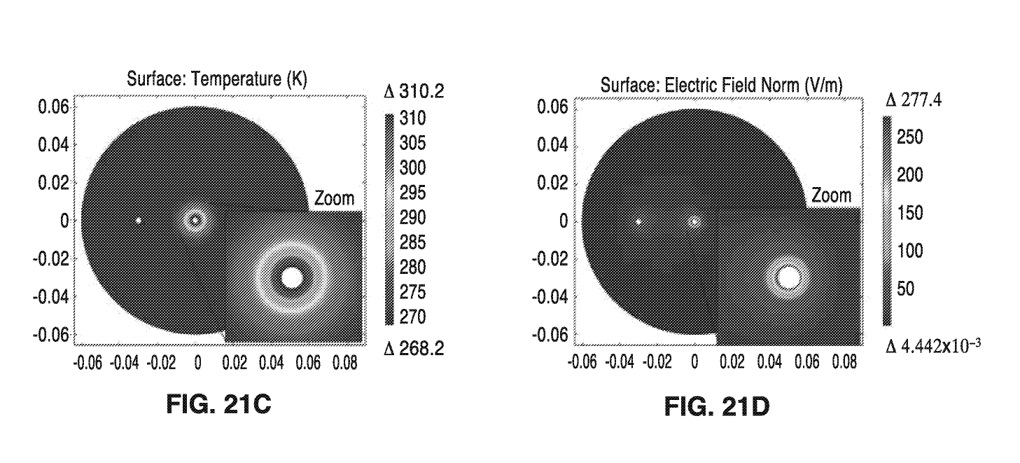

The significance of the findings in FIGS. 16A-D and 18A-D is emphasized by a comparison to the control study. The control study applies the electrical conditions on the cryoelectric probe while holding the probe and tissue at constant body temperature. The control case represents conventional PEFs delivered by a monopolar electrode. The comparison of the resulting electric fields can be seen in FIGS. 20 A-C. It is clear that the electric field in the frozen/cooled regions during freezing (FIG. 20B) and thawing (FIG. 20C) are substantially higher than the field produced in the control study in the same region (FIG. 20A). The magnitudes of the peak electric field in the freezing and thawing cases are more than double the magnitude of the peak electric field in the control case. However, at a distance from the frozen/cooled tissue, in the region of normal body temperatures, the fields during freezing and thawing are lower than those in the control. This indicates that not only do freezing/cold temperatures amplify the electric field; they also exhibit an effect of targeting the electric field to the cold region. In fact, once the electric field has decayed and reached a constant value, it is zero in the cryosurgery/PEF case, and above zero in the control PEF case.

FIGS. 20A, 20B and 20C show that the highest electric field occurs, in descending order: freezing, thawing and control. FIGS. 20A, 20B and 20C also illustrate the ability of cold/freezing temperatures to concentrate the electric field, because the most narrowly spread electric field occurs in the freezing case, followed by thawing and then control. This clearly demonstrates the ability of freezing/cold temperatures to both amplify and direct the electric fields of PEF. These results suggest the feasibility of configuring cryoelectric treatment in accordance with the present invention such that the PEF induced cell damage is confined to the frozen/cooled regions and the damage does not extend beyond these regions. The configuration of electrical parameters, which concentrate the electric field in the frozen/cooled region, also allow for cryosurgical-imaging techniques to image cells ablated due to PEFs. Ultrasound is capable of differentiating between frozen and unfrozen media during cryosurgical procedures. Therefore, by concentrating the electric field to the frozen tissue, imaging frozen tissue with ultrasound will also show cells ablated by PEFs in real time during the procedure.

As in Case 1, most temperatures in Case 2 are above −5° C. This suggests that the cryoelectric technique may not require temperatures as low as those utilized in conventional cryosurgery.

Several additional advantages of the cryosurgery/PEF procedure can be theorized as a result of these two studies. Because cells survive these high subzero freezing temperatures, cryoelectric technique would retain PEFs' ability to selectively ablate only cellular membranes without affecting the extracellular matrix. Furthermore, the cold induced electric field targeting effect may produce an additional advantage related to a major problem in PEF: electric field induced muscle contractions. The electric field in FIG. 20A has spread beyond the area in which it is effective for electroporation, while in FIGS. 20B and 20C it has not. Therefore, the targeting effect during a cryoelectric protocol holds the potential to reduce electric field induced contractions beyond the treated area.

FIGS. 20D, 20E and 20F show surface plots of the electric field distribution for the test case of FIG. 12A for the control case (FIG. 20D), freezing (FIG. 20E) and thawing (FIG. 20F).

The potential-divider circuit in FIG. 11 explains the field enhancement in regions of lower conductivity. This is because the electric field vector is normal to the boundary of the two regions of different conductivity. If the electric field is parallel to this boundary, then the equivalent circuit will be two resistors in parallel, rather than in series, and there will be negligible field enhancement or possible field decrease, depending on the relative electrical conductivity of the tissues. The particular configuration developed in this study is a direct consequence of the fact that the cryoelectric serve both as the heat sink and as the electric source. Obviously, the configurations used in this study were chosen to accomplish this effect.

Case 3:

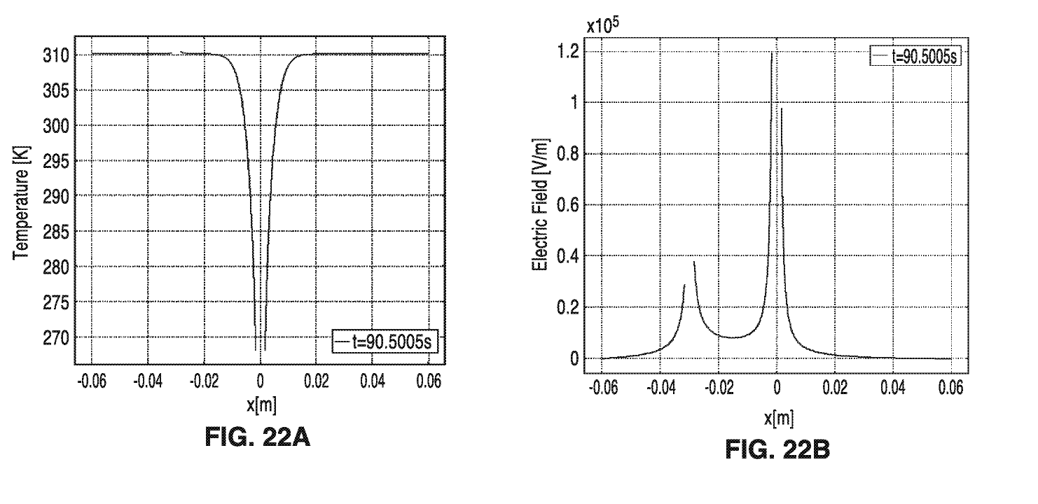

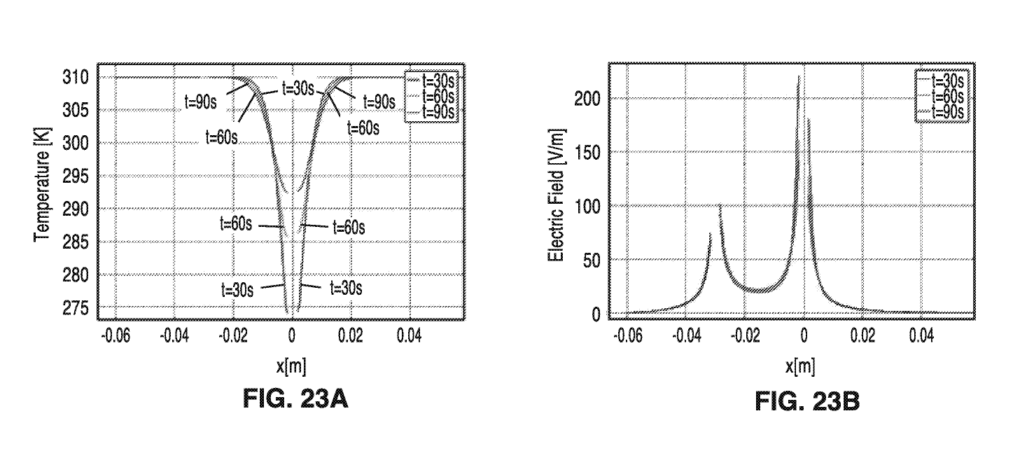

FIGS. 21A and 21B show graphs of the temperature and electric field distribution, respectively, for the test case of FIG. 12B during 90 seconds of freezing. FIGS. 21C and 21D show surface plots of the temperature and electric field distribution, respectively, for the test case of FIG. 12B during 90 seconds of freezing.

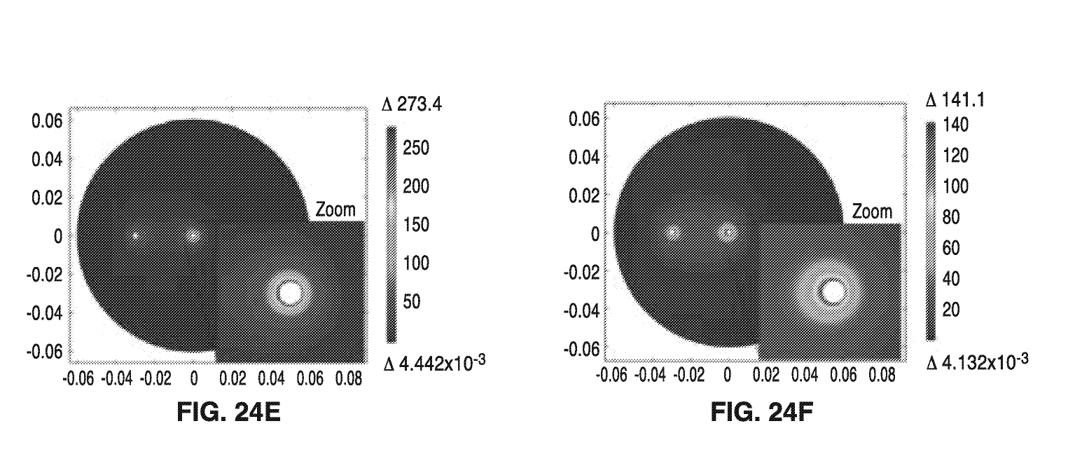

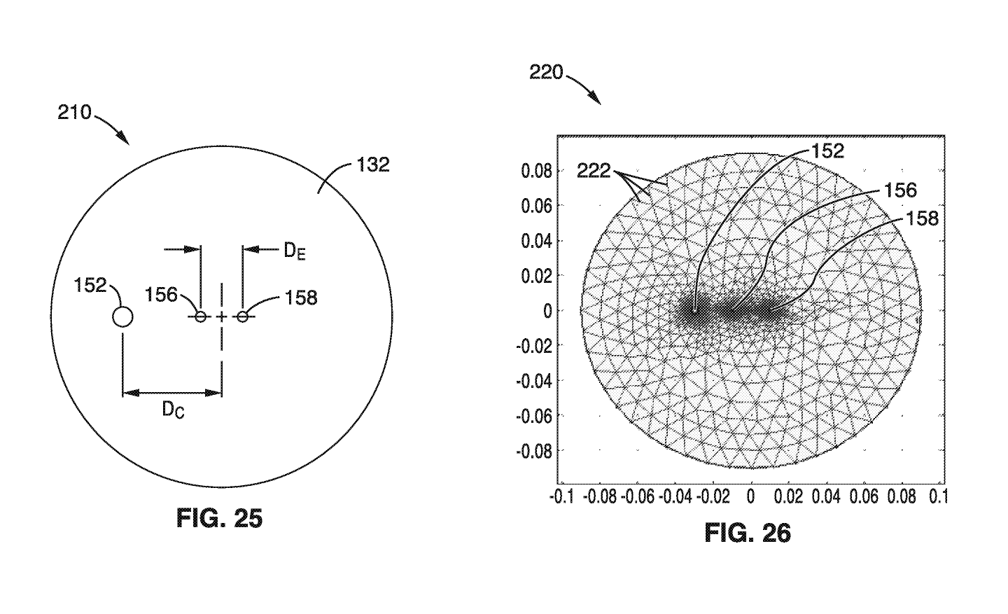

FIGS. 21A and 21C illustrate the temperature distribution which, as expected, is identical to Case 2. The electric field is plotted in FIGS. 21B and 21C. The two peaks in electric field occur adjacent to each probe 204/206. The most notable aspect of FIG. 21B is the asymmetry between these two peaks. It is shown later that in the case of the control study (FIG. 24A) the two peaks have identical magnitudes. Therefore, the asymmetry of the electric field peaks in the freezing case can be attributed to the freezing temperature applied to the rightmost probe. In this case, the application of freezing temperatures results in an electric field tripled in magnitude when only 1V is applied. FIG. 21D clearly demonstrates how the freezing/cooling concentrates the electric field to the thermally treated area. The electric field in the frozen region of the tissue is higher than 150V/m for a 1V potential on the cryo/PEF probe. This field is higher than the electric field around the unfrozen probe. Therefore, according to Eq. 6, if a potential of about 400V is set on the cryoelectric probe, only the cells in the frozen lesion will be ablated by the PEF. It should be re-emphasized that under the freezing conditions studied here, the cells in the frozen lesion would have survived freezing.

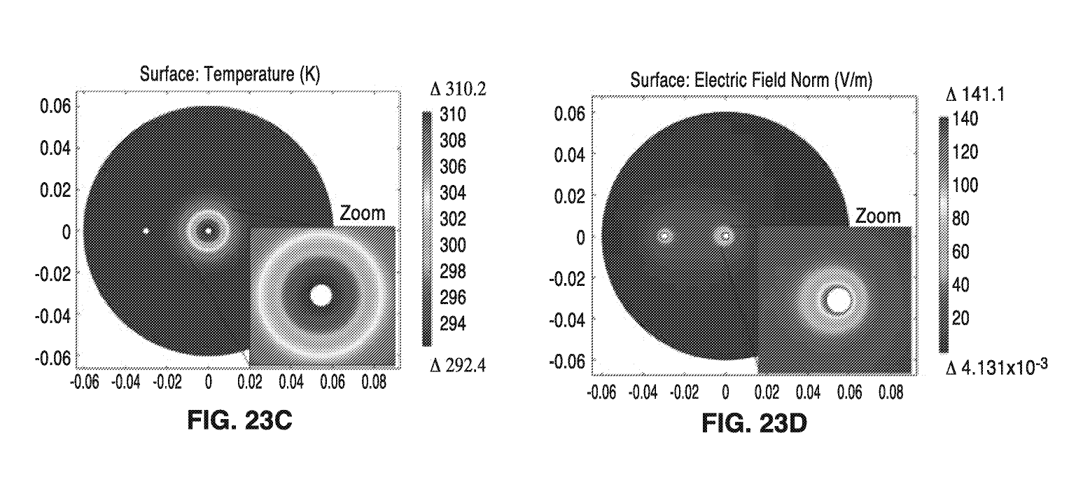

FIGS. 22A and 22B show plots of the temperature distribution and electric field distribution, respectively, after 90 seconds of freezing with an applied voltage of 400V for 90 pulses. It is clear that the higher voltage results in higher temperatures on the leftmost probe due to Joule heating, as well as higher electric fields throughout the domain.

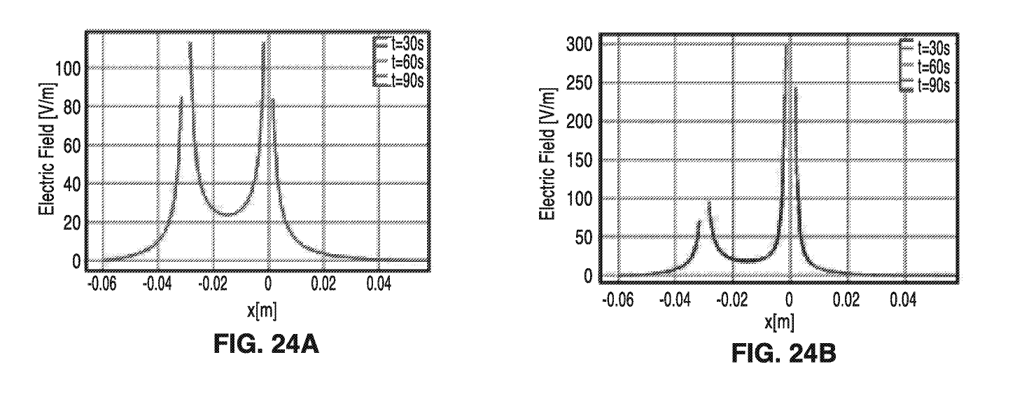

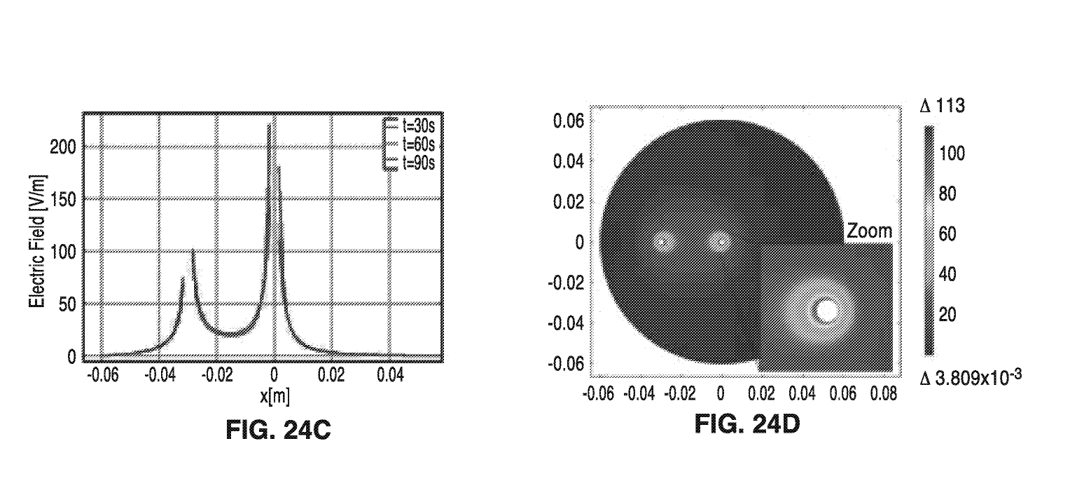

FIGS. 23A and 23B show graphs of the temperature and electric field distribution, respectively, for the test case of FIG. 12 B during 90 seconds of thawing. FIGS. 23C and 23D show surface plots of the temperature and electric field distribution, respectively, for the test case of FIG. 12 B during 90 seconds of thawing.

FIGS. 23A and 23C plot the temperature during thawing for Case 3, which is also similar to that of Case 2. The temperature distribution has a stationary point of inversion at 0.6 cm. FIG. 23B shows that the electric field also has a point of inversion around 0.6 cm, after which it drops dramatically to a constant value. The elevated temperatures in the frozen region during thawing have an effect on the electric field. As a result of higher temperatures experienced during thawing, an electric field lower than in the freezing case results. On both sides adjacent to the cryoelectric probe the electric field is lower in the thawing case than in the freezing case. After 90 seconds of thawing the peak electric field is 140V/m, while after 90 seconds of freezing the peak electric field was 275V/m. This significant decrease in resulting electric field was expected from Equation 9. FIG. 24D is similar to FIG. 21D, and clearly shows how the freezing/cooling amplifies the electric field in the thermally treated area. However, during thawing in this configuration, the electric fields in the thawing frozen region around the cryoelectric probe become comparable to those around the ground PEF probe, and irreversible electroporation pulses applied in this case may affect both the frozen and the unfrozen region. Therefore, care needs to be exercised in designing optimal cryoelectric protocols.

FIGS. 24A, 24B and 24C show the electric field distribution for the test case of FIG. 12B for the control case (FIG. 24A), freezing (FIG. 24B) and thawing (FIG. 24C). In the control, a conventional electroporation procedure was simulated, in which the electric pulse was delivered between two electrodes (probes in this case), while the temperature of the tissue and the probes were kept constant at body temperature. In the control case of FIG. 24A, the electric field around both probes is identical, as expected. In the case of thawing (FIG. 24C), the electric field is higher at the freezing probe than at the leftmost probe. And in the case of freezing (FIG. 24B), the electric field rises further on the freezing probe and decreases more on the leftmost probe. This is indicative of several facts. First of all, the drastic increase in electric field on the rightmost probe during freezing in comparison to the control study indicates that in this case, as well as Cases 1 and 2, the electric field is amplified in the frozen/cooled region. Second, the fact that the electric field is higher in the control than in the freezing case on the leftmost probe (the one without freezing applied) indicates that not only does the freezing temperature concentrate the electric field; it decreases the electric field elsewhere in the domain. This could be beneficial during treatment because it could protect surrounding structures.

The results from this part of the study show that cryoelectric protocols may be configured such that the EF induced effects on cells are confined to the frozen/cooled regions of tissue and do not extend beyond the cooled regions. Furthermore, the freezing cryoelectric probe will yield higher electric fields in the vicinity of the probe. The complementary effect of this observation is that the electric fields beyond the cooled area will be substantially reduced. Several recent studies have shown that stray electric fields beyond the PEF treated areas could have negative effects on other organs, such as the heart, or create undesirable muscle contractions. Reducing the electric fields beyond the treated area with cold should also reduce these effects. In addition, it is well established that the electric currents in PEF treatment of tissues are very high, on the order of tens of Amperes. The increased resistance caused by the cooled PEF probes, in a configuration such as the one discussed here, will substantially reduce the currents for the same applied voltage, while, on the other hand increasing the field in the cooled volume.

Note, however, that the effects discussed here, are restricted to situations in which the effect of cooling is that of a resistance in series. Next we will address a situation in which the cryocooled area is not produced by the PEF probes but rather by a different cooling probe that does not serve also as an electrode. In that case the effect is that of adding a high resistance in parallel.

D. Resistors in Parallel

In the previous examples we have shown applications of cryoelectric probes, i.e. probes that deliver both electric fields and freezing temperatures. These represent configurations shown in FIGS. 6 and 7.

The following discussion is directed to applications in which the electrodes for delivering EF are different from the freezing cryoprobe, although they operate together, as shown in FIGS. 8 and 9. The mathematical models here are the same as in the previous examples. In the analysis performed below, the freezing probe temperature was modeled as at the phase transition value.

The complex effect of temperature induced changes in tissue electrical properties can be described by voltage divider circuits consisting of elements of resistance in series or in parallel (mode 38 in FIG. 4C). The first examples dealt with freezing affected tissue resistance in series with the physiological temperature tissue impedance. The examples here deal with temperature-induced resistance in parallel with physiological temperature resistance. These two cases can also be viewed as the difference between an electrically active cooling probe (resistance in series) and an electrically inactive cooling probe (resistance in parallel). These descriptions are only an approximation of the more complex models investigated in this study, and are described here for clarification of the resulting electrical phenomena.

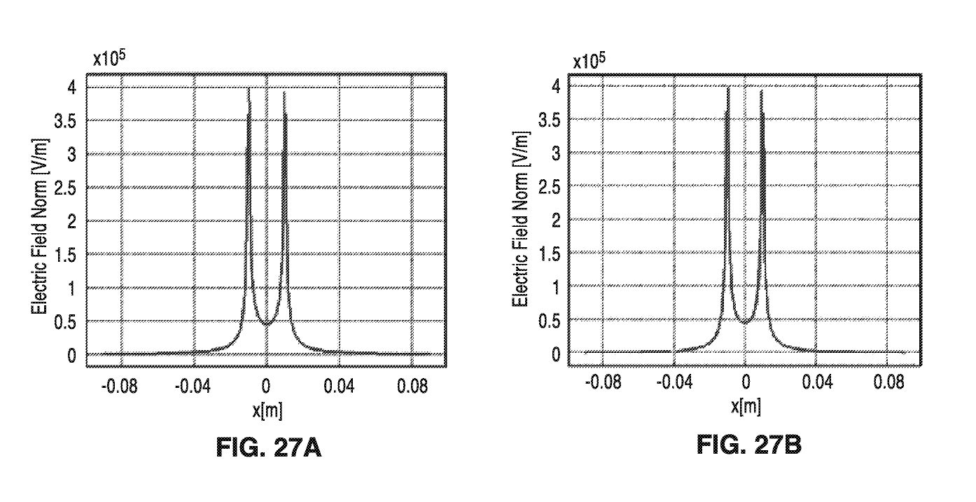

FIG. 25 is a schematic diagram of the positioning of the cooling probe 152 and electrodes 156 and 158 for a parallel circuit test setup 210. The 2D geometry utilized three probes: two electrodes 156 and 158 spaced apart at distance D_{E}, and a cooling probe 152 at a distance D_{c} from the electrodes. Because the cooling probe is not acting as an electrode and a cooling probe simultaneously, this configuration approximates two resistors in parallel.

An infinitely long cylinder of tissue 6 cm in radius was set to constant deep body temperature and electrical insulation at the outer margin. The initial temperature of the system was also deep body temperature. The delivery of PEF was applied through two typical irreversible electroporation electrodes of 1 mm in diameter. The cryoprobe was 3.4 mm in diameter. The cryoprobe 152 was set at various distances from the electrodes 156 and 158 to study the dependence of temperature induced heterogeneities in electric tissue properties on geometry. To understand the implications of temperature induced effects on the electric field, two variations have been modeled. The first applies a phase transition temperature to the cold probe. The second applies a thermally insulating boundary condition to the probe, which serves as a control.

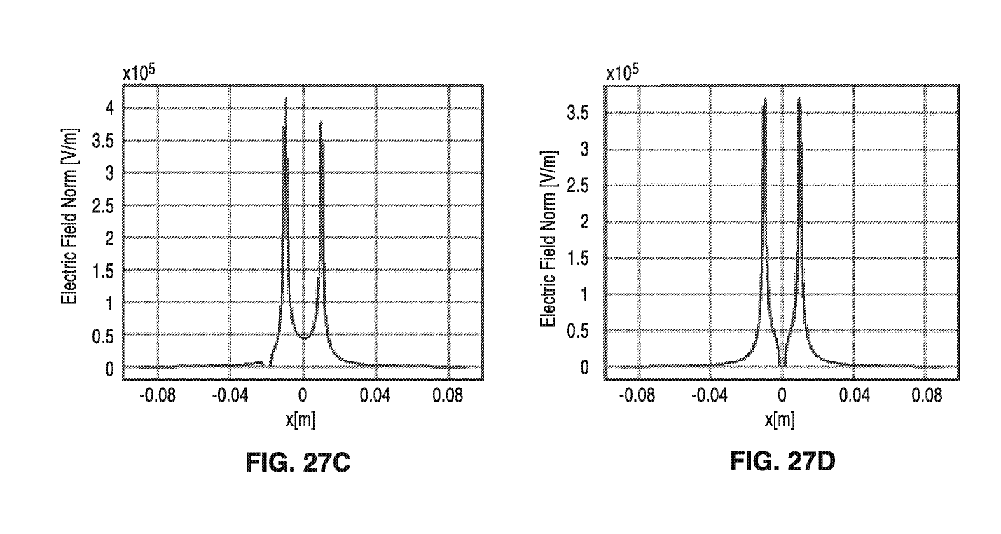

The finite element mesh 220 in this study utilized triangular elements 222, demonstrated by FIG. 26. The element size was smallest in the region surrounding the probes 152, 156 and 158. This was done in order to accurately capture the temperature gradients adjacent to both of the probes. The mesh was refined until the solution was no longer affected. Approximately 4100 elements were utilized to cover a 113 mm² surface area.

As indicated earlier, the EF's were delivered by two electrodes 156 and 158 of 1 mm diameter, separated by 2 cm. The 3.4 mm diameter cold probe 152 was placed at various locations from the center along the axial line connecting the centers of the EF electrodes 156 and 158. The protocol consisted of 2000 seconds of cooling applied by the cold probe at 0° C., followed by ten, 2500V pulses (1 Hz, 50 μs length) applied by the PEF electrodes. The cooling probe 152 was electrically insulated. In the control case, ten 2500V pulses (1 Hz, 50 μs length) applied by the PEF electrodes without using the cooling probe. Full bioheat parameters were utilized to simulate tissue.

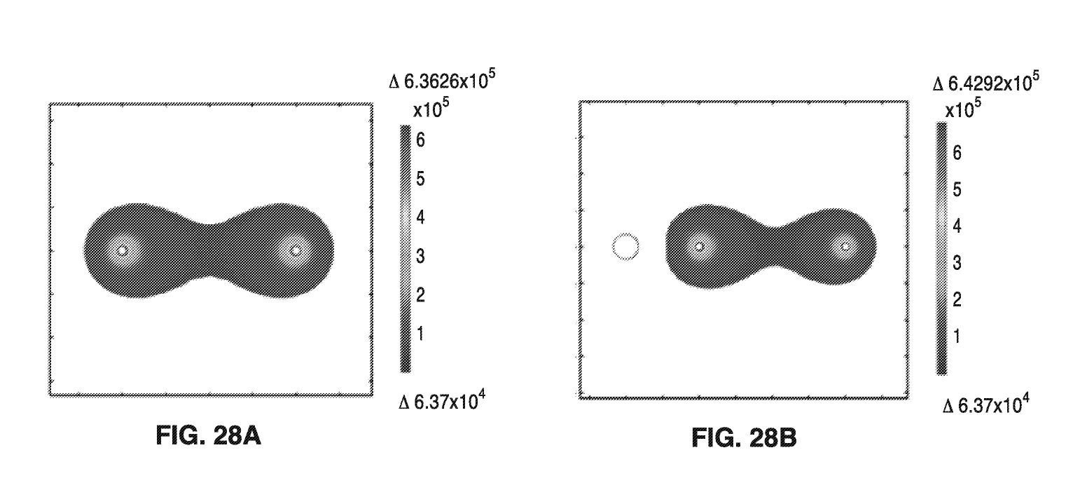

FIGS. 27A-D illustrates the electric field along a transection through the center of the electrodes and cooling probe for various locations of the cooling probe. FIG. 27A shows the control case and FIG. 27B shows the cooling case at 4 cm from the center of the domain. FIG. 27C shows the case at cold probe 2 cm from center, and FIG. 27D shows the case at cold probe 0 cm from center. It is clear that in the configuration described here the increased electric resistance due to the cold region acts as a current path of high electrical resistance in parallel to the higher temperature path of lower resistance. This configuration, in contrast to the trends identified in the resistance in series configuration results in a substantial decrease in the electric field in the cooled region. In fact, the electric field reaches 0V/m in the location of the cryoprobe probe.

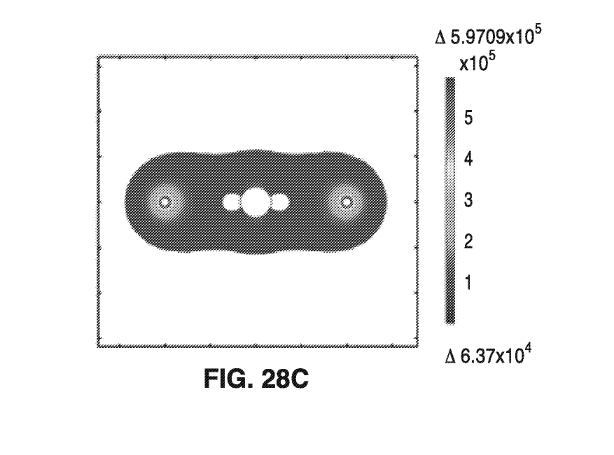

FIGS. 28A-C illustrate a potential application of this observation. Eq. 5 has been utilized to calculate the regions of tissue that undergo irreversible electroporation. The results are presented as surface plots in FIG. 28A-C (for the control case without any cooling applied (FIG. 28A), cooling probe located 2 cm from the center (FIG. 28B), and cooling probe located at the center of the tissue (FIG. 28C)).

FIG. 28A-C demonstrate that a cryoprobe operating at high subzero temperatures can avoid irreversible electroporation and freezing damage at a particular location. FIG. 28B demonstrates that this can be accomplished outside of the treatment region when the cooling probe is placed at a distance from the electrodes. FIG. 28C demonstrates that a cryoprobe can protect a region between the two electrodes as well.

These results illustrate a potential important application of the use of freezing in EF's. When comparing the top and middle panels, it is seen that in the middle panel the temperature induced changes in electrical potential due to the cold probe cause the irreversible electroporation field near the probe to recede (the edge flattens). This suggests that if a sensitive tissue structure is close to the outer edge of the PEF fields of treatment, the simple application of cold can protect this structure. The bottom panel shows that the use of cold can eliminate PEF's even in the center of the treated area. This suggests that any critical tissues, which need protection during a PEF protocol, can be protected by cold and high subzero freezing. One clinically relevant example is the bladder sphincter that is particularly vulnerable during the IRE treatment of the prostate. Freezing the sphincter to high subzero temperatures and thereby increasing its electrical resistance could protect it from damage during minimal invasive treatment of the prostate with IRE. This could be achieved, for instance with a catheter through the urethra that is insulated in such a way that it freezes only the sphincter area.

The goal of this study was to evaluate the characteristics of a combination freezing and electric fields application on tissue. Perhaps the most important conclusion from this study is that the combination of freezing and electric fields can produce applications that are not feasible with each of the modalities alone.

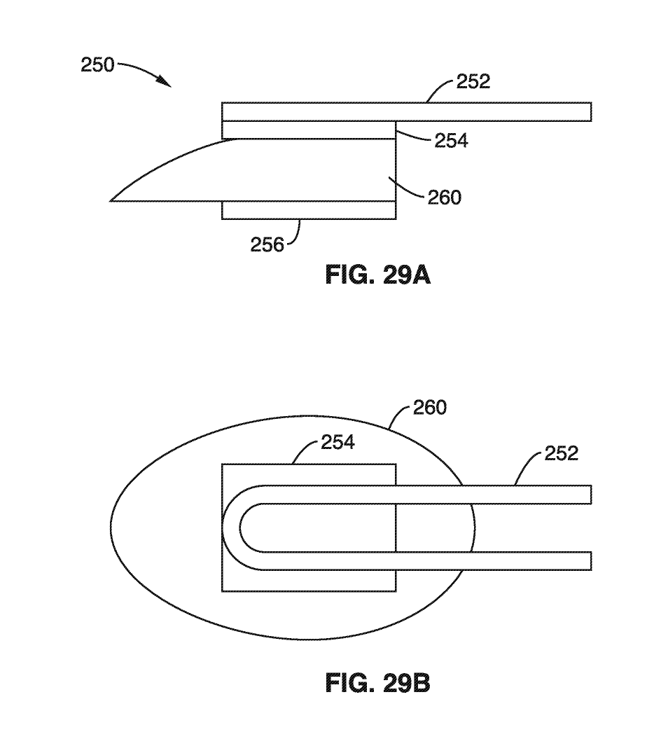

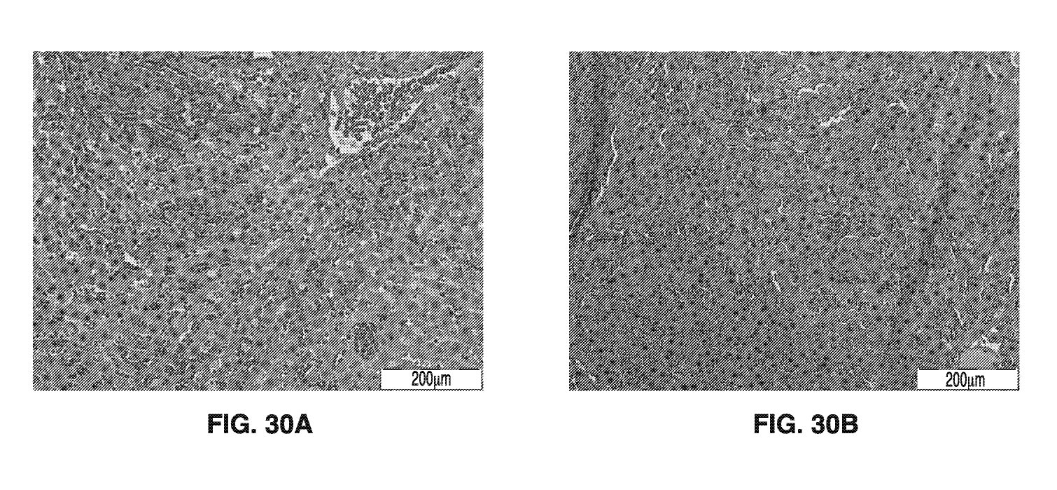

Referring now to FIGS. 29A-B and FIGS. 30A-B additional in vivo experiments were performed to study characteristics of the cryoelectric systems and methods of the present invention, and in particular: a) the ability to apply electric fields through frozen tissue, b) the enhanced cell death when a combination of freezing and application of electric pulses is applied from the same cryoelectric probe, and c) the tight sticking of the cryoelectric probe to tissue upon freezing.

FIGS. 29A and 29B show side and top schematic views, respectively, of a test setup 250 performed on liver tissue in vivo 160. Cryoelectric electrodes 254 and 256 were attached across one lobe of liver 260.

The electrodes 254 and 256 comprised 20 mm by 20 mm by 2 mm thick copper plates that clamp the liver 260. The electrodes 254, 256 were connected with electric wires to a BTX 830 (Harvard instruments Boston Mass.) electroporation device (not shown). The top electrode 254 was welded to a 6 mm outer diameter 5 mm inner diameter copper cooling tube 252 whose ends were connected with Teflon tubing to a Neslab RT 140 cooling system (not shown) that uses an alcohol as the cooling fluid. The top electrode 254 temperature was measured with a T type thermocouple (not shown).

A cryoelectric protocol in accordance with the present invention was compared against a standard cryo protocol. In the cryoelectric protocol, the liver 260 was clamped between the setup 250 of FIGS. 29A and 29B, and the Neslab cooling system generated flow of cooling fluid through the copper tube 252 until a temperature of −5 C was measured on the top electrode 254. After five minutes at this temperature, an electric pulse sequence of 70 pulses 100 microseconds long was delivered at a frequency of 4 Hz and with an electric field of 2000 V/cm. In the cryo protocol the experiment was similar in all the parameters of cryo and the time except no electric pulses were delivered.

Two hours after the application of the pulses the liver lobe was excised and the treated tissue stained with H&E and examined histologically.

Several key observations were made from this experiment. First, it was observed that the freezing produced sticking between the top electrode 254 and the tissue 260. To release the tight connection between the top electrode 254 and the tissue 260, it was necessary to warm up the interface either by pouring warm saline on the system or by perfusing the copper tubes 252 with warm saline. Furthermore, it was observed that the muscular contractions normally observed during the delivery of PEF to the intestine did not occur during the cryoelectric procedure.

FIGS. 30A and 30B show comparisons of the histology of the cryo-only treated tissue (FIG. 30A) and tissue treated with the device shown in FIGS. 29A-B (FIG. 30B). The histology shown in FIG. 30A shows that cells survive freezing in the cryo-only protocol, and many cells have fully developed nuclei and intact cell membranes.

In contrast, the histology shown in FIG. 30B shows that the combination cryoelectric produces complete cell death as evidence by the picknotic (dark) nuclei in all the cells and lack of clear cell membrane.

The histology provides evidence to two facts: a) electric pulses can be delivered through frozen tissue, and b) the combination cryo and electric pulses is much more effective in destroying cells than cryo alone.

The above examples are directed primarily to the effects of PEF in combination with tissue temperatures that are lowered to a level at or below the freezing temperature of the tissue. While there are significant benefits to cooling the tissue to high sub-freezing temperatures, it is appreciated that PEF's in combination with tissue temperatures lower than the physiological body temperature, but above the level of freezing, may also have beneficial affects for treatment of various conditions.

From the discussion above it will be appreciated that the invention can be embodied in various ways, including the following:

1. A method for treating a target volume of biological matter, comprising: cooling a volume of tissue to a temperature below freezing; and directing an electric field through the cooled volume of tissue or tissue adjacent to said cooled volume of tissue to generate at least a temporary physiological affect on one or more of the cooled volume of tissue and adjacent volume of tissue.

2. The method of embodiment 1: wherein the target volume comprises the adjacent volume of tissue; and wherein the cooled volume of tissue is substantially shielded from the electric field while being directed through the adjacent volume of tissue.

3. The method of embodiment 1: wherein the target volume comprises the cooled volume of tissue; and wherein the electric field is configured to destroy cells within the cooled volume of tissue that would otherwise survive freezing.

4. The method of embodiment 3, wherein the electric field is configured to sterilize the cooled volume of tissue.

5. The method of embodiment 1, wherein cooling a volume of tissue and directing an electric field further comprises: inserting a first probe within the target volume of tissue; the first probe being coupled to a cooling source; directing coolant through the first probe to cool the cooled volume of tissue within a region surrounding or adjacent to the first probe; the first probe further being coupled to an EF source to form a first electrode; coupling a second electrode to the EF source, the second electrode being positioned at or within tissue external to the cooled volume of tissue; and directing the electric field between the first and second electrodes.

6. The method of embodiment 5, wherein the electric field is substantially concentrated in the cooled volume of tissue to irreversibly destroy tissue cells only within the cooled volume of tissue.

7. The method of embodiment 5, wherein the first and second electrodes form a series circuit with the cooled volume of tissue and adjacent volume of tissue.

8. The method of embodiment 5, wherein the first probe is configured to simultaneously cool the cooled volume of tissue and propagate the electric field through the cooled volume of tissue.

9. The method of embodiment 2, wherein cooling a volume of tissue and directing an electric field further comprises: inserting a cooling probe within the volume of tissue; coupling first and second electrodes to tissue adjacent the volume of tissue such that the cooling probe is positioned between the first and second electrodes; cooling the volume of tissue with the cooling probe; and directing the electric field between the first and second electrodes.

10. The method of embodiment 9: wherein the first and second electrodes comprise first and second electrode probes; and wherein the first and second electrode probes are inserted into adjacent volumes of tissue external to the cooled volume of tissue.

11. The method of embodiment 9, wherein the first and second electrodes form a circuit in parallel with the cooled volume of tissue and adjacent volumes of tissue.

12. The method of embodiment 9, wherein the electric field is concentrated in the adjacent volumes of tissue to substantially shield the cooled volume of tissue from the electric field.

13. The method of embodiment 1, wherein the cooled volume of tissue is cooled to a temperature ranging between −190° C. and 0° C.

14. The method of embodiment 13, wherein the cooled volume of tissue is cooled to a temperature ranging between −30° C. and 0° C.

15. The method of embodiment 14, wherein the cooled volume of tissue is cooled to a temperature ranging between −10° C. and 0° C.

16. The method of embodiment 15, wherein the cooled volume of tissue is cooled to a temperature ranging between −5° C. and −0.56° C.

17. The method of embodiment 1, wherein the electric field comprises a pulsed electric field.

18. The method of embodiment 17, wherein the electric field comprises an alternating current producing electric field.

19. The method of embodiment 17, wherein the pulsed electric field is configured to affect cells in one or more of the cooled volume of tissue and adjacent volume of tissue via reversible electroporation.

20. The method of embodiment 17, wherein the pulsed electric field is configured to induce cell damage in one or more of the cooled volume of tissue and adjacent volume of tissue via irreversible electroporation.

21. The method of embodiment 17, wherein the electrical field is applied at 100 V/cm or greater.

22. The method of embodiment 21, wherein the electrical field is applied at a range of 100 V/cm to 100,000 V/cm.

23. The method of embodiment 22, wherein the electrical field is applied at a range of 100 V/cm to 5000 V/cm.

24. The method of embodiment 22, wherein the electrical field is applied at a pulse length ranging from 1 nanosecond to 10 milliseconds.

25. The method of embodiment 24, wherein the electrical field is applied at a pulse length ranging from 10 microseconds to 200 microseconds.

26. The method of embodiment 22, wherein the electrical field is applied with a number of pulses ranging from 1 to 1000.

27. The method of embodiment 26, wherein the electrical field is applied with a number of pulses ranging from 5 to 100.

28. The method of embodiment 22, wherein the electrical field is applied at a frequency of AC ranging from 0.5 Hz to 10⁹ Hz.

29. The method of embodiment 28, wherein the electrical field is applied at a frequency of AC ranging from 0.5 Hz to 10 kHz

30. The method of embodiment 25, wherein the electrical field is applied at an interval between pulses ranging from 1 microsecond to 10 seconds.

31. The method of embodiment 30, wherein the electrical field is applied at an interval between pulses ranging from 100 microseconds to 1 second.

32. A method as recited in embodiment 4, wherein the electric field is delivered to the frozen volume tissue in a series of 10 kV/cm to 40 kV/cm pulses.

33. The method of embodiment 1, further comprising: modulating the electric field in the cooled volume of tissue as a function of the temperature in the cooled volume of tissue.

34. The method of embodiment 1, wherein cooling a volume of tissue comprises placing a cooling probe adjacent a tissue surface to freeze at least a portion of the tissue surface such that the cooling probe sticks by freezing to the portion of the tissue surface; and directing an electric field through the cooling probe to treat tissue at or near the tissue surface.

35. The method of embodiment 1, wherein cooling a volume of tissue comprises inserting a cooling probe into tissue to freeze at least a portion of the tissue such that the cooling probe sticks by freezing to the tissue; and directing an electric field through the cooling probe to treat tissue at or near the site of probe insertion in tissue.

36. The method of embodiment 1, further comprising: imaging with a medical imaging device the cooled volume of tissue and adjacent volume of tissue to identify the extent of concentrated electric field delivery within the biological matter.

37. The method of embodiment 36, further comprising: identifying tissue cells damaged by the electric field by identifying the cooled volume of tissue.

38. A system for treating a target volume of biological matter, comprising: a first probe; a coolant source coupled to the first probe for delivering coolant to the first probe; the first probe configured for cooling a volume of tissue to a temperature below freezing; and one or more electrodes configured to be electrically coupled to the biological matter; the one or more electrodes coupled to an EF source for directing an electric field through the cooled volume of tissue or tissue adjacent to said cooled volume of tissue to generate at least a temporary physiological affect on one or more of the cooled volume of tissue and adjacent volume of tissue.

39. The system of embodiment 38, wherein the first probe is electrically coupled to an EF source to form a first electrode, further comprising: a second electrode coupled to the EF source; wherein the second electrode is configured to be positioned at or within tissue external to the cooled volume of tissue to direct an electric field between the first and second electrodes; wherein the first and second electrodes are configured to generate a confined physiological affect on the cooled volume of tissue.

40. The system of embodiment 39, wherein the first and second electrodes are configured to induce cell damage within the cooled volume of tissue.

41. The system of embodiment 40, wherein the first and second electrodes form a series circuit with the cooled volume of tissue and adjacent volume of tissue.

42. The system of embodiment 39, wherein the first probe is configured to simultaneously cool the target volume of tissue and propagate the electric field through the target volume of tissue.

43. The system of embodiment 39: wherein first probe comprises a conductive material having a distal end and proximal end; wherein the proximal end comprises an insulative layer such that the cooling and electric field are only propagated from the distal end of the first probe.

44. The system of embodiment 39: wherein first probe comprises a probe surface configured to contact a tissue surface associated with the cooled volume of tissue; wherein the probe surface is configured to stick to the tissue surface to engage the cooled volume of tissue prior to delivery of the electric field to the cooled volume of tissue.

45. The system of embodiment 39: wherein first probe comprises a probe surface configured to be inserted in a tissue associated with the cooled volume of tissue; wherein the probe surface is configured to stick to the tissue to engage the cooled volume of tissue prior to delivery of the electric field to the cooled volume of tissue.

46. The system of embodiment 38: wherein the first probe comprises a cooling probe configured to be positioned within the volume of tissue; the one or more electrodes comprising first and second electrodes configured to be coupled to tissue adjacent the volume of tissue on opposing sides of the cooling probe is positioned between the first and second electrodes; wherein the cooling probe and first and second electrodes are configured to concentrate the electric field in adjacent tissue to substantially shield the cooled volume of tissue from the electric field.

47. The system of embodiment 46, wherein the first and second electrodes form a circuit in parallel with the cooled volume of tissue and adjacent tissue.

48. The system of embodiment 46: wherein the first and second electrodes comprise first and second electrode probes; and wherein the first and second electrode probes are inserted into adjacent volumes of tissue external to the cooled volume of tissue

49. The system of embodiment 38, wherein the EF source is configured to deliver a pulsed electric field.

50. The system of embodiment 49, wherein the pulsed electric field is configured to induce cell damage in one or more of the cooled volume of tissue and adjacent volume of tissue via irreversible electroporation.

51. The system of embodiment 49, wherein the pulsed electric field is configured to induce changes in cells in one or more of the cooled volume of tissue and adjacent volume of tissue via reversible electroporation.

52. An apparatus treating a target volume of tissue, comprising: a cryoelectric probe; a coolant source coupled to the cryoelectric probe for delivering coolant to the cryoelectric probe; the cryoelectric probe configured for cooling the target volume of tissue to a temperature below freezing; the cryoelectric probe further being electrically coupled to an EF source to form a first electrode; and a second electrode coupled to the EF source; wherein the second electrode is configured to be positioned at or within tissue external to the cooled volume of tissue to direct an electric field between the first and second electrodes; wherein the first and second electrodes are configured to generate a confined physiological affect on the cooled volume of tissue and the adjacent tissue.

53. An apparatus as recited in embodiment 52, wherein the first and second electrodes are configured to induce cell damage within the cooled volume of tissue.

54. An apparatus as recited in embodiment 53, wherein the first and second electrodes form a series circuit with the cooled volume of tissue and adjacent volume of tissue.

55. An apparatus as recited in embodiment 52, wherein the cryoelectric probe is configured to simultaneously cool the target volume of tissue and propagate the electric field through the target volume of tissue.

56. An apparatus as recited in embodiment 52; wherein the cryoelectric probe comprises a conductive material having a distal end and proximal end; wherein the proximal end comprises an insulative layer such that the cooling and electric field are only propagated from the distal end of the first probe.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1

| | | Blood | | | Tissue |
|---|---|---|---|---|---|
| Thermal | Blood | Heat | Metabolic | Tissue | Heat |
| Conductivity | Perfusion | Capacity | Heat | Density | Capacity |
| 0.5 | 0.5 | 3640 | 33800 | 1000 | 3750 |
| [W/mK] | [kg/m³s] | [J/kgK] | [W/m³] | [kg/m³] | [J/kgK] |

TABLE 2

| | Density | | Thermal |
|---|---|---|---|
| | (ρ) | Specific Heat | Conductivity (k) |
| | [kg/m³] | (Cₚ) [J/kg K] | [W/m K] |
| Frozen | 918 | 2052 | 2.31 |
| Unfrozen | 997 | 4179 | 0.613 |

What is claimed is:

1. A method for treating a target volume of biological matter, comprising:
cooling a volume of tissue to a temperature below freezing; and
directing an electric field through the cooled volume of tissue or tissue adjacent to said cooled volume of tissue to generate at least a temporary physiological effect on one or more of the cooled volume of tissue and adjacent volume of tissue.

2. A method as recited in claim 1, wherein cooling a volume of tissue and directing an electric field further comprises:
inserting a first probe within the target volume of tissue;
the first probe being coupled to a cooling source;
directing coolant through the first probe to cool the cooled volume of tissue within a region surrounding or adjacent to the first probe;
the first probe further being coupled to an EF source to form a first electrode;
coupling a second electrode to the EF source, the second electrode being positioned at or within tissue external to the cooled volume of tissue; and
directing the electric field between the first and second electrodes.

3. A method as recited in claim 1, wherein cooling a volume of tissue and directing an electric field further comprises:
inserting a cooling probe within the volume of tissue;
coupling first and second electrodes to tissue adjacent the volume of tissue such that the cooling probe is positioned between the first and second electrodes;
cooling the volume of tissue with the cooling probe; and
directing the electric field between the first and second electrodes.

4. A method as recited in claim 1, further comprising: modulating the electric field in the cooled volume of tissue as a function of the temperature in the cooled volume of tissue.

5. A method as recited in claim 1, wherein cooling a volume of tissue comprises placing a cooling probe adjacent a tissue surface to freeze at least a portion of the tissue surface such that the cooling probe sticks by freezing to the portion of the tissue surface; and
directing an electric field through the cooling probe to treat tissue at or near the tissue surface.

6. A method as recited in claim 1, wherein cooling a volume of tissue comprises inserting a cooling probe into tissue to freeze at least a portion of the tissue such that the cooling probe sticks by freezing to the tissue; and
directing an electric field through the cooling probe to treat tissue at or near the site of probe insertion in tissue.

7. A method as recited in claim 1, further comprising:
imaging with a medical imaging device the cooled volume of tissue and adjacent volume of tissue to identify the extent of concentrated electric field delivery within the biological matter.

8. A method as recited in claim 7, further comprising:
identifying tissue cells damaged by the electric field by identifying the cooled volume of tissue.

9. A system for treating a target volume of biological matter, comprising:
a first probe;
a coolant source coupled to the first probe for delivering coolant to the first probe;
the first probe configured for cooling a volume of tissue to a temperature below freezing; and
one or more electrodes configured to be electrically coupled to the biological matter;
the one or more electrodes coupled to an EF source for directing an electric field through the cooled volume of tissue or tissue adjacent to said cooled volume of tissue to generate at least a temporary physiological affect on one or more of the cooled volume of tissue and adjacent volume of tissue.

10. A system as recited in claim 9, wherein the first probe is electrically coupled to an EF source to form a first electrode, further comprising:
a second electrode coupled to the EF source;
wherein the second electrode is configured to be positioned at or within tissue external to the cooled volume of tissue to direct an electric field between the first and second electrodes; and
wherein the first and second electrodes are configured to generate a confined physiological affect on the cooled volume of tissue.

11. A system as recited in claim 10:
wherein first probe comprises a conductive material having a distal end and proximal end; and
wherein the proximal end comprises an insulative layer such that the cooling and electric field are only propagated from the distal end of the first probe.

12. A system as recited in claim 10:
wherein first probe comprises a probe surface configured to contact a tissue surface associated with the cooled volume of tissue; and
wherein the probe surface is configured to stick to the tissue surface to engage the cooled volume of tissue prior to delivery of the electric field to the cooled volume of tissue.

13. A system as recited in claim 10:
wherein first probe comprises a probe surface configured to be inserted in a tissue associated with the cooled volume of tissue; and
wherein the probe surface is configured to stick to the tissue to engage the cooled volume of tissue prior to delivery of the electric field to the cooled volume of tissue.

14. A system as recited in claim 9:
wherein the first probe comprises a cooling probe configured to be positioned within the volume of tissue;
the one or more electrodes comprising first and second electrodes configured to be coupled to tissue adjacent the volume of tissue on opposing sides of the cooling probe is positioned between the first and second electrodes; and
wherein the cooling probe and first and second electrodes are configured to concentrate the electric field in adjacent tissue to substantially shield the cooled volume of tissue from the electric field.

15. A system as recited in claim 14:
wherein the first and second electrodes comprise first and second electrode probes; and
wherein the first and second electrode probes are inserted into adjacent volumes of tissue external to the cooled volume of tissue

16. An apparatus treating a target volume of tissue, comprising:
a cryoelectric probe;
a coolant source coupled to the cryoelectric probe for delivering coolant to the cryoelectric probe;
the cryoelectric probe configured for cooling the target volume of tissue to a temperature below freezing;
the cryoelectric probe further being electrically coupled to an EF source to form a first electrode; and
a second electrode coupled to the EF source;
wherein the second electrode is configured to be positioned at or within tissue external to the cooled volume of tissue to direct an electric field between the first and second electrodes;
wherein the first and second electrodes are configured to generate a confined physiological affect on the cooled volume of tissue and the adjacent tissue.

17. An apparatus as recited in claim 16, wherein the first and second electrodes are configured to induce cell damage within the cooled volume of tissue.

18. An apparatus as recited in claim 17, wherein the first and second electrodes form a series circuit with the cooled volume of tissue and adjacent volume of tissue.

19. An apparatus as recited in claim 16, wherein the cryoelectric probe is configured to simultaneously cool the target volume of tissue and propagate the electric field through the target volume of tissue.

20. An apparatus as recited in claim 16;
wherein the cryoelectric probe comprises a conductive material having a distal end and proximal end; and
wherein the proximal end comprises an insulative layer such that the cooling and electric field are only propagated from the distal end of the first probe.
